(19) United States
(12) Patent Application Publication
(10) Pub. No.: US 2013/0078676 A1
(43) Pub. Date: Mar. 28, 2013

(54) METHOD OF REGULATING OLIGONUCLEOTIDE FUNCTIONALITY

(71) Applicants: Alexander Alan Morley, Malvern (AU); Michael Julian Brisco, Campbelltown (AU)

(72) Inventors: Alexander Alan Morley, Malvern (AU); Michael Julian Brisco, Campbelltown (AU)

(73) Assignees: FINDERS UNIVERSITY, BEDFORD PARK (AU); MONOQUANT PTY LTD, ADELAIDE (AU)

(21) Appl. No.: 13/637,342

(22) PCT Filed: Feb. 25, 2011
(86) PCT No.: PCT/AU11/00218

§ 371 Date: Nov. 28, 2012

Related U.S. Application Data
(60) Provisional application No. 61/319,146, filed on Mar. 30, 2010.

(51) Int. Cl. C12P 19/34

(52) U.S. Cl. CPC C12P 19/34
USPC 435/91.2

(57) ABSTRACT

The present invention relates generally to a method of regulating oligonucleotide functionality and, more particularly, to a method of regulating the functionality of a primer or probe. The method of the invention is designed to provide a means to selectively inactivate or activate the functionality of an oligonucleotide, such as a primer, thereby providing means to regulate the progress of any method using that oligonucleotide. The development of a means to regulate the functionality of an oligonucleotide, such as a primer, is useful in a range of applications including, but not limited to, amplification reactions such as PCR, isothermal amplification and nucleic acid strand extension. With respect to amplification reactions, these have wide utility including the diagnosis and/or monitoring of disease conditions which are characterised by specific gene sequences and the characterisation or analysis of specific gene regions of interest.

### FIELD OF THE INVENTION

The present invention relates generally to a method of regulating oligonucleotide functionality and, more particularly, to a method of regulating the functionality of a primer or probe. The method of the invention is designed to provide a means to selectively inactivate or activate the functionality of an oligonucleotide, such as a primer, thereby providing means to regulate the progress of any method using that oligonucleotide. The development of a means to regulate the functionality of an oligonucleotide, such as a primer, is useful in a range of applications including, but not limited to, amplification reactions such as PCR, isothermal amplification and nucleic acid strand extension. With respect to amplification reactions, these have wide utility including the diagnosis and/or monitoring of disease conditions which are characterised by specific gene sequences and the characterisation or analysis of specific gene regions of interest.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Bibliographic details of the publications referred to by author in this specification are collected alphabetically at the end of the description.

The polymerase chain reaction (PCR) is a technique which is utilised to amplify specific regions of a DNA strand. This may be a single gene, just a part of a gene or a non-coding sequence. Most PCR methods typically amplify DNA fragments of up to 10 kilo base pairs (kb), although some techniques allow for amplification of fragments up to 40 kb in size (Cheng et al., 1994, Proc Natl Acad Sci. 91:5695-5699).

PCR, as currently practiced, requires several basic components (Sambrook and Russel, 2001, Molecular Cloning: A Laboratory Manual, 3^{rd} Ed.). These components are:
a DNA template which contains the region of the DNA fragment to be amplified;
oligonucleotides, which act as primers and which are complementary to the DNA regions at the 5’ and 3′ ends of the DNA region that is to be amplified;
a DNA polymerase (e.g. Taq polymerase or another thermostable DNA polymerase with a temperature optimum at around 70° C.), used to synthesize a DNA copy of the region to be amplified; and
Deoxynucleotide triphosphates (dNTPs) from which the DNA polymerase builds the new DNA.

PCR is carried out in small reaction tubes (0.2-0.5 ml volumes), containing a reaction volume typically of 15-100 μl, which are inserted into a thermal cycler. This machine heats and cools the reaction tubes within it to the precise temperature required for each step of the reaction. Most thermal cyclers comprise heated lids to prevent condensation on the inside of the reaction tube caps. Alternatively, a layer of oil may be placed on the reaction mixture to prevent evaporation.

Accordingly, PCR is a method that allows exponential amplification of DNA sequences within a longer DNA molecule. The reaction involves a number of cycles of amplification, and in each cycle the template for each molecular reaction is either a strand of the initial DNA in the sample or a strand of DNA synthesised in a preceding cycle. Each PCR cycle involves the following steps
denaturation by heat to separate the 2 strands of the DNA duplex
hybridisation of the upstream and downstream primers to their complementary sequences
extension of the primers by the DNA polymerase to produce a complementary copy of the template sequence

Typically the PCR reagents and conditions are chosen so that denaturation, hybridisation and extension occur at close to maximum efficiency and as a result the amount of the desired sequence increases with each cycle by a factor of close to 2. Substantial amplification occurs by the end of the PCR eg a 30 cycle PCR will result in amplification of the original template by a factor of almost 2³⁰ (1,000,000,000). This degree of amplification facilitates detection and analysis of the amplified product.

After a number of cycles of amplification, the PCR may be terminated and the product analysed in various ways, most commonly by gel electrophoresis. When the PCR is carried out using a fixed number of cycles, the amount of amplified product is usually not closely related to the amount of input target DNA, and this type of PCR is rather a qualitative tool for detecting the presence or absence of a particular DNA and/or for providing sufficient target DNA for further analysis.

In order to measure messenger RNA (mRNA), the method uses reverse transcriptase to initially convert mRNA into complementary DNA (cDNA) which is then amplified by PCR and analyzed by agarose gel electrophoresis. Reverse transcription followed by end-point PCR is similarly essentially a qualitative technique.

In order to provide quantification capability, real-time PCR was developed. This procedure follows the general pattern of PCR, but the amplified DNA is quantified during each cycle. Two common methods of quantification are the use of fluorescent dyes that intercalate with double-stranded DNA and modified DNA oligonucleotide primers or probes the fluorescence of which changes during one of the steps of the PCR. Frequently, real-time polymerase chain reaction is combined with reverse transcriptase polymerase chain reaction to quantify low abundance messenger RNA (mRNA), enabling a researcher to quantify relative gene expression at a particular time or in a particular cell or tissue type.

(i) Real-Time PCR Using Dyes Binding to Double-Stranded DNA

A DNA-binding dye, such as Sybr Green, binds to all double-stranded (ds)DNA in a PCR reaction, causing increased fluorescence of the dye. An increase in DNA product during PCR therefore leads to an increase in fluorescence intensity which is measured at each cycle, thus allowing DNA concentrations to be quantified.

(ii) Fluorescent Reporter Sequence Methods

A number of different methods using fluorescent reporter primers or probes have been developed and they tend to be more accurate and reliable than use of DNA binding dyes. They use one or more DNA primers or probes to quantify only the DNA to which the primer or probe hybridises. Use of a reporter probe, such as a Taqman probe, significantly increases specificity and may allow quantification even in the presence of some non-specific DNA amplification. Use of sequence-specific primers or probes allows for multiplexing—assaying for several different amplified products in the same reaction by using specific sequences or probes with different-coloured labels, provided that all targets are amplified with similar efficiency.

In terms of quantification, relative concentrations of DNA present during the exponential phase of the reaction are determined by plotting fluorescence against cycle number on a logarithmic scale. A threshold for increase of fluorescence above background or decrease below background (depending on the precise method) is determined. The cycle at which the fluorescence from a sample crosses the threshold is called the cycle threshold, Cₜ.

The amount of target DNA is then determined by comparing the test results to the results produced by one or more standards. When the target DNA is genomic DNA, then a series of standards, usually 10 fold dilutions of a known amount of the target DNA, are commonly used. When the target DNA is cDNA, then one or more internal standards of the cDNA of another gene are commonly used.

A variation of traditional PCR, designed to increase the specificity of the PCR amplification, is the nested PCR reaction. In this amplification reaction, two sets of primers are used in two successive reactions. In the first, one pair of primers is used to generate DNA products, which may also contain products amplified from non-target areas. The products from the first PCR are then used to start a second, using one (‘hemi-nesting’) or two different primers whose binding sites are located (nested) within the first set. The specificity of all of the primers is combined, usually leading to a single product.

Nested PCR is conventionally performed by carrying out an initial PCR in one reaction tube, transferring an aliquot of the amplified products into a second reaction tube, and then carrying out a second PCR. This procedure has two disadvantages. It is more complex than a single PCR and, more importantly, it carries the risk of contaminating the environment with the amplified products of the first PCR, which may lead to contamination of subsequent experimental procedures. For this reason, several methods have been developed for carrying out the successive PCRs in the one reaction tube.

Carrying out two rounds of PCR in the one reaction tube involves adding the primers for the two rounds into the initial reaction mixture. The methods that have then been used for producing two sequential rounds of PCR, the first using the outer pair of primers and the second using the inner pair, include:
using different annealing temperatures for the two rounds of PCR (Kemp et al., Gene 1990, 94:223-228, Erlich et al. U.S. Pat. No. 5,314,809),
decreasing the concentration of the primers for the first round PCR (Erlich et al. U.S. Pat. No. 5,314,809)
varying the annealing times for the two rounds of PCR Grosz et al U.S. Pat. No. 5,340,728)
modifying the structure of the primers for the second round of PCR and using two different annealing temperatures during this round. (Xu Dingbang Publication CN1858219).
using a low denaturation temperature for the second round PCR (Erlich et al U.S. Pat. No. 5,314,809)
using chemically modified primers for the first round PCR and an enzyme which would progressively destroy them (Du Breuil Lastrucci U.S. Pat. No. 7,273,730)
using an initial physical separation of the reagents for the first and second round PCRs (Yourno U.S. Pat. No. 5,556,773, Ching et al. United States Patent Application 20060177844).

The principle underlying all of these methods is to produce, at some point, a rapid or gradual loss of the activity of the primers for the first round PCR so that ongoing amplification depends progressively on the activity of the primers for the second round PCR. However, all of these methods have disadvantages, the nature of the disadvantage depending upon the method. Their robustness varies and the reaction conditions may need to be adjusted depending on the sequence of interest which is to be amplified. Amplification may be inefficient, in some cases throughout the first round PCR and in other cases during the transition from the first round to the second round PCR. As a consequence, some of the approaches are not widely used in practice, whereas others are used only for detection and not for quantification.

Most amplification methods which are routinely performed rely on thermal cycling to achieve amplification. There have also been developed a range of isothermal amplification techniques, such as transcription mediated amplification, strand displacement amplification, loop mediated isothermal amplification, isothermal multiple displacement amplification and helicase dependent amplification. However, these isothermal amplification reactions tend to be more complex than the polymerase chain reaction and somewhat more difficult to optimise. They therefore tend to be used more for end-point detection rather than for quantification. They do nonetheless have the advantage that a thermal cycler is not required thereby making these methods significantly simpler to set up. This can be a significant issue in environments where access to sophisticated equipment, such as thermal cyclers, is not possible. It is also possible to perform nested isothermal amplification although this is not often done. These various isothermal reactions are either completely DNA-based or at some stage involve cyclical production of RNA, but they all involve the use of DNA primers which hybridise and extend under the action of a DNA polymerase.

In work leading up to the present invention, there has been developed a method of regulating oligonucleotide functionality, such as primer functionality, in terms of both its activation and inactivation, based on the use of antisense oligonucleotides. This development has enabled improvement in the efficiency of, for example, primer-based technologies such as nucleic acid amplification. For example, the method of the present invention has enabled the development of a single tube nested PCR in which specificity and efficiency are both improved based on the ability to control primer functionality. This enables efficient amplification using selected primers followed by efficient amplification using other primers. This amplification method is particularly useful due to its utility with both isothermal and thermal nucleic acid amplification reactions. However, the present invention is useful beyond merely its application in the context of amplification reactions and extends to utility with respect to any reaction in which the activity of an oligonucleotide is required to be modulated during the course of the reaction.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method of modulating the functionality of an oligonucleotide of interest, said method comprising:
(i) contacting said oligonucleotide of interest with an antisense oligonucleotide directed to said oligonucleotide of interest and effecting a nucleotide sequence change in either or both of the oligonucleotide of interest or the antisense oligonucleotide wherein said sequence change modulates the functionality of the oligonucleotide of interest; or
(ii) effecting a sequence change in an oligonucleotide complex, which complex comprises an oligonucleotide of interest hybridised to an antisense oligonucleotide and wherein said sequence change is effected in an oligonucleotide which forms part of said complex and modulates the functionality of the oligonucleotide of interest.

In another embodiment the present invention is directed to a method of modulating the capacity of a primer to undergo extension along a target nucleic acid, said method comprising:
(i) contacting said primer with an antisense oligonucleotide directed to said primer and effecting a nucleotide sequence change in either or both of the primer or the antisense oligonucleotide wherein said sequence change modulates primer functionality; or
(ii) effecting a sequence change in an oligonucleotide complex, which complex comprises a primer hybridised to an antisense oligonucleotide directed to said primer and wherein said sequence change is effected in an oligonucleotide which forms part of said complex and modulates primer functionality.

A further aspect of the present invention is directed to a method of modulating the capacity of an oligonucleotide of interest to undergo extension along a target nucleic acid, said method comprising hybridising said oligonucleotide to the 3′ end of an antisense oligonucleotide and facilitating the 3′ extension of said oligonucleotide of interest along said antisense oligonucleotide wherein the antisense nucleotide sequence along which said oligonucleotide of interest extends generates an extension of said oligonucleotide which is either:
(i) complementary to the nucleotide sequence of the target nucleic acid and thereby renders said oligonucleotide of interest functional;
(ii) mismatched relative to the nucleotide sequence of the target nucleic acid and thereby renders said oligonucleotide of interest non-functional; or
(iii) complementary to a nucleotide sequence of another region of said oligonucleotide of interest and wherein said extension hybridises to said region of said oligonucleotide of interest thereby forming a stem-loop configuration and rendering said oligonucleotide of interest non-functional.

In another aspect there is provided a method of modulating the functionality of an oligonucleotide of interest, said method comprising:
(i) hybridising an antisense oligonucleotide to the 3′ end of said oligonucleotide of interest and facilitating the 3′ extension of said antisense oligonucleotide along said oligonucleotide of interest thereby rendering said oligonucleotide of interest non-functional; or
(ii) hybridising the 3′ end of an antisense oligonucleotide to the 3′ end of said oligonucleotide of interest and facilitating the 3′ extension of said antisense oligonucleotide along said oligonucleotide of interest and the 3′ extension of said oligonucleotide of interest along said antisense oligonucleotide thereby rendering said oligonucleotide of interest non-functional; or
(iii) contacting an oligonucleotide complex, which oligonucleotide complex comprises an oligonucleotide of interest hybridised to an antisense oligonucleotide, with a primer which hybridises to said antisense oligonucleotide at a region which is 3′ to the region to which the oligonucleotide of interest is hybridised and effecting 3′ extension of said primer wherein extending said primer displaces said oligonucleotide of interest and renders said oligonucleotide of interest functional; or
(iv) extending the linear 3′ strand of a looped antisense oligonucleotide, which 3′ linear strand is hybridised to a 5′ linear strand but which 5′ linear strand is longer than the 3′ linear strand and comprises a single stranded region which is complementary to said oligonucleotide of interest and which is hybridised to said oligonucleotide of interest, wherein extending said 3′ linear strand along said single stranded region displaces said oligonucleotide of interest and renders said oligonucleotide of interest functional; or
(v) selectively degrading the linear antisense strand either of a stem-loop nucleic acid molecule or of a duplex between said linear antisense strand and an oligonucleotide of interest, which degradation renders the complementary linear oligonucleotide of interest strand single-stranded and thereby functional; or
(vi) hybridising oligonucleotide of interest with a low Tm to an antisense oligonucleotide and effecting extension of said oligonucleotide of interest wherein said extended oligonucleotide of interest develops an increased Tm and is thereby rendered functional; or
(vii) contacting an oligonucleotide complex, which oligonucleotide complex comprises an oligonucleotide of interest hybridised to the 3′ end of an antisense oligonucleotide and a second oligonucleotide hybridised to said antisense oligonucleotide 3′ to the region to which the oligonucleotide of interest is hybridised and effecting ligation between said oligonucleotide of interest and said second oligonucleotide wherein the functionality of said oligonucleotide of interest is modulated.

In one embodiment, said nucleic acid is DNA.

In another embodiment said oligonucleotide of interest is directed to a target molecule which is either a protein or a nucleic acid molecule.

In another embodiment functionality of said oligonucleotide of interest is functionality as a primer, aptamer, DNAzyme, RNase or ribozyme.

In another embodiment there is provided a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed; and
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable extension of said first primers due to antisense oligonucleotide hybridisation to said first primers.

In still another embodiment the present invention is directed to a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable and which antisense oligonucleotide directed to said first primer comprises a 5′ nucleic acid tag sequence, the complementary nucleotide sequence of which tag is mismatched relative to the nucleotide sequence of the DNA region adjacent to the 5′ end of the hybridisation site of said primer; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said first primer along said tagged antisense oligonucleotide.

In yet another embodiment the present invention is directed to a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable and which antisense oligonucleotide directed to said first primer comprises a 5′ nucleic acid tag sequence, the complementary nucleotide sequence of which tag is complementary relative to the nucleotide sequence of another region of the same primer; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said first primer along said tagged antisense oligonucleotide.

In yet still another embodiment there is provided a method of amplifying a target DNA, said method comprising:
(i) contacting a DNA sample with:
   (a) a first forward primer directed to said target DNA and a first reverse, primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable and one or more of which antisense oligonucleotides hybridise to the 3′ end of said first primer and are extendible in the 3′ direction along said primer; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said antisense oligonucleotide along said primer.

In still yet another embodiment there is provided a method of amplifying a target DNA t, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed
      and which second primers comprise a 3′ tag which is mismatched relative to the nucleotide sequence of the target DNA; and
   (c) one or more antisense oligonucleotides as defined hereinbefore directed to one or more of said primers wherein the functionality of said primer is thereby modulatable and one of said antisense oligonucleotide sequences comprises both a 3′ region complementary to the 3′ region of said second primer and a more 5′ region the complementary sequence of which is complementary relative to the sequence of the target DNA adjacent to the primer binding site; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which enable hybridisation and extension of said second primers along said antisense oligonucleotide; and
(iv) amplifying the DNA sample of step (iii) under conditions which enable hybridisation and extension of said second primers along the DNA region of interest but which do not enable extension of said first primers due to antisense oligonucleotide hybridisation to said first primers.

### BRIEF DESCRIPTION OF THE DRAWINGS

FIG. 1 is a schematic representation of use of an antisense oligonucleotide to inactivate a primer. During the course of the reaction, either due to a change in annealing temperature or to the course of time during a constant annealing temperature, the antisense oligonucleotide and the primer hybridise, the primer extends to generate a mismatch to its template which prevents extension.

FIG. 2 is a schematic representation of another use of an antisense oligonucleotide to inactivate a primer. During the course of the reaction, either due to a change in annealing temperature or to the course of time during a constant annealing temperature, the antisense oligonucleotide and the primer hybridise, the primer extends to generate a sequence which is complementary to an upstream sequence of the primer. The single stranded extended primer forms a stem-loop structure with the stem extending to the 5′ end of the primer and this inhibits hybridisation to the template.

FIG. 3 is a schematic representation of use of an oligonucleotide which undergoes extension and converts an active primer to an inactive primer. Either due to a change in temperature conditions or to the passage of time during a constant annealing temperature, hybridisation of the primer and antisense molecules occurs. The hybridised antisense molecules extend, the Tm increases as does their ability to bind primer molecules. With the passage of time the concentration of free primer molecules decreases to the point at which their function declines.

FIG. 4 is a schematic representation of use of an antisense oligonucleotide with a 5′ tag in which both the primer and the antisense oligonucleotide undergo extension and the primer is converted to an inactive primer. Either due to a change in temperature conditions or to the passage of time during a constant annealing temperature, hybridisation of the primer and antisense molecules occurs. Both the primer and the antisense molecules extend, the Tm of each increases and hybridisation between them becomes very strong so that the concentration of free primer decreases markedly. To ensure that extension of the primer does not result in increased binding to its template, the sequence of the 5′ tag on the antisense oligonucleotide must be, such that extension of the primer results in a mismatch to its template.

FIG. 5 is a schematic representation of use of an antisense oligonucleotide to activate an inactive primer. The primer is initially inactive owing to the presence of two or more nucleotides at its 3′ end which are mismatched to the template. When the antisense oligonucleotide and primer hybridise, the primer extends along the oligonucleotide tag, the sequence of which is such that the sequence of the extension of the primer is now a perfect match to the template.

FIG. 6 is a schematic representation of the use of an additional displacing primer in order to convert a primer from an inactive state to an active state. Initially the primer is inactive owing to being bound to the antisense oligonucleotide. However, with the passage of time or with change in annealing temperature, the displacing primer binds to the antisense oligonucleotide, extends, and displaces the primer so that the concentration of free primer increases. The free primer is then able to bind to its template.

FIG. 7 is a schematic representation of the use of an antisense oligonucleotide to convert a primer from an inactive to an active configuration. In this case primer molecules with a low Tm are initially free, not bound to either the template onto the antisense oligonucleotide. Either due to a change in temperature conditions or to the passage of time during a constant annealing temperature, some primer molecules hybridise to the antisense oligonucleotide at their 3′ end. The primer molecules extend and the sequence of the antisense oligonucleotide is such that the extended primer is a perfect match to the template. Following denaturation of the duplex, the extended primer preferentially hybridises to the template rather than to the antisense oligonucleotide.

FIG. 8 is a schematic representation of the use of an antisense oligonucleotide to convert a primer from an inactive to an active configuration. The antisense oligonucleotide and the primer oligonucleotide sequences may be separate molecules which are hybridised to form a duplex (a) or they may be part of a single stem-loop molecule (b). The antisense moiety is potentially degradable. Initially, the primer sequence is strongly hybridised to the antisense sequence. With the passage of time, degradation of the antisense sequence occurs progressively and, the primer sequence is now free and able to hybridise to its template. One mechanism of degradation is the presence within the antisense of one or more RNA nucleotides and, optionally, the presence of a ribonuclease in the reaction mixture.

FIG. 9 is a schematic representation of the use of extension of an antisense oligonucleotide to convert a primer from an inactive state to an active state. Initially the primer is inactive owing to being bound to the antisense oligonucleotide. Most of the time the rudimentary stem-loop of the antisense molecule is in the open position. However, with the passage of time or with change in annealing temperature, in an increasing number of antisense molecules the rudimentary stem closes, extension occurs and the concentration of free primer molecules increases to the point that efficient hybridisation to template and efficient amplification occur.

FIG. 10 is a schematic representation of the use of a ligation reaction to modulate the function of an oligonucleotide of interest. The antisense molecule acts as a template to enable hybridisation of the oligonucleotide of interest and a second oligonucleotide. Ligation of the latter 2 oligonucleotides then occurs and the thereby-produced extension on the oligonucleotide of interest results in modulation of the function of the oligonucleotide of interest.

FIG. 11 is a graphical representation of an experiment studying hybridisation of a primer to itself to form a stem-loop followed by extension. The experiment illustrated the concept shown in FIG. 2. A series of “extended” primers were synthesised such that the 3′ end consisted of 4-12 bases which could potentially hybridise to a more 5′ region of the primer and thereafter extend such that the final hybridised region could comprise up to 12 bases. To enable hybridisation and extension to occur, the primers were subjected to 12 cycles of PCR and a melting analysis was performed on the resulting products. Sybr Green was present in the melting analysis so that the double-stranded DNA of the stem could be recognised by the fluorescence which results from intercalation of this agent. The temperature at which the stem of the stem-loop dissociates is related to the length of the stem. It can be seen from the identical temperature disassociation peaks that the primers which initially comprised a hybridising region ranging from 6-12 bases all extended during the PCR to form a stem-loop structure with a stem of 12 bases. However the primers which comprised a hybridising region of 4 or 5 bases did not extend during the PCR, presumably because hybridisation was ineffective.

FIG. 12 is an image of results of an experiment in which an antisense oligonucleotide was used to produce single-stranded DNA. In this experiment 2 primers (12-75 and 13-75) were used to amplify a region of the gene for the SNf2 related CREBBP activator protein in the presence or absence of one of two antisense oligonucleotides (12AS or 13AS) which were designed to hybridise to and inactivate the 12-75 primer. Initially there were 15 PCR cycles performed at 72° C., during which exponential amplification occurred. The PCR then continued for a further 43 cycles at 58° C., during which the antisense oligonucleotide bound to the 12-75 primer and produced progressive inactivation. However continuing extension of the 13-75 primer at each round produced increasing amounts of single-stranded DNA. This is seen as the upper band in the figure. There is a small amounts of single-stranded DNA present after a large number of cycles even when the antisense oligonucleotide is absent (left panel). This presumably reflects some degree of primer imbalance with using up of one of the primers.

FIG. 13. Mechanism of antisense PCR. Phase 1 has a high annealing temperature; only the longer outer primers can bind to target and generate product. Phase 2 has a lower annealing temperature, and two processes occur. First, the antisense oligonucleotide anneals to the outer primer, the outer primer extends along the oligonucleotide, and the 3 ′ end of the outer primer consequently becomes mismatched to the amplicon template and the primer is inactivated. Second, the shorter inner primer can now bind to the target and extend. This progressively switches the PCR from using the outer primers to using the inner primers,

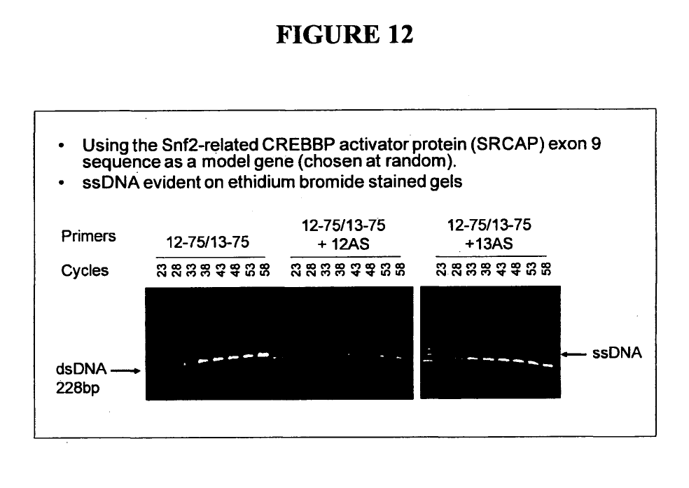

FIG. 14. Analysis of antisense qPCR studying four different genes. Amplification was by (i) the outer primers alone; (ii) the outer primers plus the antisense oligonucleotides, showing that the oligonucleotides inhibit amplification; and (iii) the outer primers plus the antisense oligonucleotides plus the inner primers, showing that the inner primers restore amplification. The Ct of 55 indicates no detectable signal by the end of the PCR.

FIG. 15. Analysis of antisense PCR by electrophoresis. In this experiment, 10 ng of DNA was amplified for 15 high-temperature cycles followed by 20 low-temperature cycles. Amplification with only the outer primers produced the expected 333-bp band, and that with the complete antisense system produced the expected 111-bp directed by the inner primers. The weak band in tracks B and E is consistent with a 158-bp product directed by one outer primer and one inner primer. 1˜ and 2× refer to amounts being one or two times the usual protocol amount of 100 ng for each inner primer, 200 ng for each inner primer, and an amount of antisense equimolar to the corresponding outer primer.

FIG. 16. Analysis of APC amplification products during antisense PCR. A primary PCR was performed using two-phase conditions and either the outer primers (OP), the antisense oligonucleotides (AS), and the inner primers (IP); the outer primers only; or the outer primers plus antisense oligonucleotides. After 15, 18, 21, 24, or 32 cycles, the PCR was stopped and an aliquot was assayed in a secondary PCR to measure the products formed during the primary PCR. For antisense PCR, amplification could be due to outer primers or inner primers; thus, either long products or the total of long and short products were measured by using either the outer primers or the inner primers, respectively. In the other two PCRs, inner primers were absent, so only long products were measured. In the data legend, the description to the left of the slash indicates the constituents in the primary PCR and that to the right indicates the primers, and hence the product measured, in the secondary PCR. Note that the Cₜ values are shown inversely to illustrate the increasing amount of product generated with increasing cycles of the PCR. There is an exponential increase both in the total number of amplicons in the antisense PCR and in the number of long amplicons in the PCR where only the outer primers are present (the regression lines for both sets of data are shown). However, when antisense oligonucleotides are present, amplification of long amplicons gradually slows both in the antisense PCR and in the PCR where the only primers present are the long ones.

FIG. 17. Decreased nonspecific amplification during antisense PCR. For two genes, N-RAS and BCR, amplification was performed by either antisense PCR or standard PCR using the outer primers, and fluorescence was measured either specifically by a Taqman probe or nonspecifically by SYBR Green. For antisense PCR, there was no significant difference between the Ct measured by the two endpoints, whereas for standard PCR, the Cₜ measured by SYBR Green was significantly less than that measured by Taqman, indicating the generation of substantially more nonspecific amplification.

### DETAILED DESCRIPTION OF THE INVENTION

The present invention is predicated, in part, on the design of antisense technology which enables the inducible inactivation or activation of one or more oligonucleotides which participate in a reaction. For example, the method of the present invention is very useful when the oligonucleotides are primers in the context of a nested amplification reaction where one can design a single tube nested PCR method which maintains a constant and optimal level of efficiency. This development has enabled a degree of control and robustness not previously available in the context of primer-based technologies. Still further, in the context of amplification methods this method is applicable to both thermal and isothermal nucleic acid amplification reactions.

Accordingly, one aspect of the present invention is directed to a method of modulating the functionality of an oligonucleotide of interest, said method comprising:
(i) contacting said oligonucleotide of interest with an antisense oligonucleotide directed to said oligonucleotide of interest and effecting a nucleotide sequence change in either or both of the oligonucleotide of interest or the antisense oligonucleotide wherein said sequence change modulates the functionality of the oligonucleotide of interest; or
(ii) effecting a sequence change in an oligonucleotide complex, which complex comprises an oligonucleotide of interest hybridised to an antisense oligonucleotide and wherein said sequence change is effected in an oligonucleotide which forms part of said complex and modulates the functionality of the oligonucleotide of interest.

Reference to an “oligonucleotide of interest” should be understood as a reference to any DNA or RNA molecule in respect of which functionality is sought to be regulated or modified. To this end, reference to “functionality” should be understood as a reference to the ability of the oligonucleotide to perform an action on or in association with a target molecule e.g. by acting as a primer which undergoes extension, by hybridising to a nucleic acid target or other target molecule, or by acting as a nucleic acid enzyme. Reference to “modulate” should be understood as referring to either the acquisition of a new function of the oligonucleotide or the effecting of a change in the level of a pre-existing function, which may be achieved by effecting a change in the concentration of free (i.e. unhybridised) oligonucleotide or by a change in its sequence or structure. It should be appreciated that to the extent that the oligonucleotide of interest is a primer, in some scenarios the primer may hybridise to its target nucleic acid but if it cannot extend, then that primer is regarded as non-functional in the context of the present invention. The oligonucleotide of interest is one which, in the context of its functionality, is required, among other things, to hybridise to a complementary molecular region, most commonly a target nucleic acid region. This may occur in the context of a wide variety of different types of reactions including, but not limited to, probe based technologies (such as Southern blotting), primer based technologies (such as nucleic acid amplification), nucleic acid extension, and reactions which involve the oligonucleotide binding to and/or modifying a target (such as when the oligonucleotide of interest acts as an aptamer, DNAzyme or ribozyme). In one embodiment, said oligonucleotide of interest is a primer.

According to this embodiment the present invention is directed to a method of modulating the capacity of a primer to undergo extension along a target nucleic acid, said method comprising:
(i) contacting said primer with an antisense oligonucleotide directed to said primer and effecting a nucleotide sequence change in either or both of the primer or the antisense oligonucleotide wherein said sequence change modulates primer functionality; or
(ii) effecting a sequence change in an oligonucleotide complex, which complex comprises a primer hybridised to an antisense oligonucleotide directed to said primer and wherein said sequence change is effected in an oligonucleotide which forms part of said complex and modulates primer functionality.

Reference to a “target molecule”, should be understood as a reference to a molecule to which said oligonucleotide of interest hybridises or binds. The target molecule may be any non-proteinaceous or proteinaceous molecule, such as a nucleic acid or protein. It should be understood, however, that a target nucleic acid molecule is not the antisense oligonucleotide or another primer which uses said antisense oligonucleotide of interest as a template for extension or ligation.

To the extent that the target molecule is a nucleic acid, such as a nucleic acid which is sought to be amplified, reference to a nucleic acid “region of interest” or “target nucleic acid” should be understood as a reference to any region of DNA or RNA which is sought to be amplified or probed. This may be a gene, part of a gene or an intergenic region. To this end, reference to “gene” should be understood as a reference to a DNA molecule which codes for a protein product, whether that be a full length protein or a protein fragment. In terms of chromosomal DNA, the gene will include both intron and exon regions. However, to the extent that the DNA of interest is cDNA, such as might occur if the DNA of interest is vector DNA or reverse transcribed mRNA, there may not exist intron regions. Such DNA may nevertheless include 5′ or 3′ untranslated regions. Accordingly, reference to “gene” herein should be understood to encompass any form of DNA which codes for a protein or protein fragment including, for example, genomic DNA and cDNA. The subject nucleic acid region of interest may also be a non-coding portion of genomic DNA which is not known to be associated with any specific gene (such as the commonly termed “junk” DNA regions). It may be any region of genomic DNA produced by recombination, either between 2 regions of genomic DNA or 1 region of genomic DNA and a region of foreign DNA such as a virus or an introduced sequence. It may be a region of a partly or wholly synthetically or recombinantly generated nucleic acid molecule. The subject nucleic acid region of interest may also be a region of DNA which has been previously amplified by any nucleic acid amplification method, including polymerase chain reaction (PCR) (i.e. it has been generated by an amplification method).

The subject “nucleic acid” region or “oligonucleotide” may be DNA or RNA or derivative or analogue thereof. Where the region of interest is a DNA sequence which encodes a proteinaceous molecule it may take the form of genomic DNA, cDNA which has been generated from a mRNA transcript, or DNA generated by nucleic acid amplification. However where the subject DNA does not encode a protein, either genomic DNA or synthetically or recombinantly generated DNA may be the subject of analysis. As would be appreciated by the skilled person, both synthetically and recombinantly generated DNA may also encode all or part of a protein. However, if the subject method is directed to an RNA region of interest, it would be appreciated that it will usually first be necessary to reverse transcribe the RNA to DNA, such as using RT-PCR. The subject RNA may be any form of RNA, such as mRNA, primary RNA transcript, ribosomal RNA, transfer RNA, micro RNA or the like. Preferably, said nucleic acid region of interest is a DNA region of interest. To this end; said DNA includes DNA generated by reverse transcription from RNA which is ultimately the subject of analysis, and DNA generated by a nucleic acid amplification method such as PCR.

Accordingly, another aspect of the present invention is directed to a method of modulating the functionality of a DNA oligonucleotide of interest, said method comprising:
(i) contacting said oligonucleotide of interest with an antisense oligonucleotide directed to said oligonucleotide of interest and effecting a nucleotide sequence change in either or both of the oligonucleotide of interest or the antisense oligonucleotide wherein said sequence change modulates the functionality of the oligonucleotide of interest; or
(ii) effecting a sequence change in an oligonucleotide complex, which complex comprises an oligonucleotide of interest hybridised to an antisense oligonucleotide and wherein said sequence change is effected in an oligonucleotide which forms part of said complex and modulates the functionality of the oligonucleotide of interest.

In one embodiment, said oligonucleotide of interest is directed to a target molecule which is either a protein or a nucleic acid molecule.

In another embodiment, said oligonucleotide of interest is a primer, aptamer, DNAzyme, ribozyme or RNase.

The present invention more particularly provides a method of modulating the capacity of a primer to undergo extension along a target DNA, said method comprising:
(i) contacting said primer with an antisense oligonucleotide directed to said primer and effecting a nucleotide sequence change in either or both of the primer or the antisense oligonucleotide wherein said sequence change modulates primer functionality; or
(ii) effecting a sequence change in an oligonucleotide complex, which complex comprises a primer hybridised to an antisense oligonucleotide directed to said primer and wherein said sequence change is effected in an oligonucleotide which forms part of said complex and modulates primer functionality.

Reference to “DNA” should be understood as a reference to deoxyribonucleic acid or derivative or analogue thereof. In this regard, it should be understood to encompass all forms of DNA, including cDNA and genomic DNA. The nucleic acid molecules of the present invention may be of any origin including naturally occurring (such as would be derived from a biological sample), recombinantly produced or synthetically produced.

Reference to “derivatives” should be understood to include reference to fragments, homologs or orthologs of said DNA from natural, synthetic or recombinant sources. “Functional derivatives” should be understood as derivatives which exhibit any one or more of the functional activities of DNA. The derivatives of said DNA sequences include fragments having particular regions of the DNA molecule fused to other proteinaceous or non-proteinaceous molecules. “Analogs” contemplated herein include, but are not limited to, modifications to the nucleotide or nucleic acid molecule such as modifications to its chemical makeup or overall conformation. This includes, for example, incorporation of novel or modified purine or pyrimidine bases or modification to the manner in which nucleotides or nucleic acid molecules interact with other nucleotides or nucleic acid molecules such as at the level of backbone formation or complementary base pair hybridisation. The biotinylation or other form of labelling of a nucleotide or nucleic acid molecules is an example of a “functional derivative” as herein defined.

Preferably, said DNA is a gene or gene fragment, a chromosomal gene translocation breakpoint or DNA produced by prior nucleic acid amplification, such as PCR. The DNA of interest may be chemically synthesised or may be derived from the DNA or RNA of any organism including, but not limited to, any animal, plant, bacterium or virus.

Without limiting the present invention to any one theory or mode of action, it has been determined that oligonucleotide functionality can be regulated via antisense technology. Specifically, a method for either inactivating and/or thereafter activating the functionality of an oligonucleotide, such as a primer, has been developed based on the use of antisense molecules together with effecting a sequence change in either the oligonucleotide or the antisense molecule such that the functionality of the oligonucleotide (e.g. the capacity of a primer to hybridise or undergo extension) is modulated. Reference to “extension” in this context should be understood as a reference to the ability of that primer to be induced by a polymerase to undergo 3′ nucleic acid extension along a template to which it is hybridised. Such extension reactions are predominantly utilised in the context of nucleic acid amplification reactions such as PCR. However, it would be appreciated by the person of skill in the art that there are also other applications for primer extension reactions, such as production of single stranded DNA. The sequence change can be effected by any suitable means including, for example, the use of antisense molecules which directly block the capacity of an oligonucleotide to extend subsequently to its hybridisation to a target (or conversely can be degraded to reveal a functional primer) or which themselves undergo extension or form the template for oligonucleotide extension in order to similarly regulate the functionality of the primer.

In classical PCR, the primers and reaction conditions are designed so that hybridisation and extension of the forward and reverse primers occur at or close to maximum efficiency so that the number of amplicons approximately doubles with each cycle, resulting in efficient exponential amplification. An adequate concentration of primers is important in achieving optimal efficiency. Greater specificity of DNA amplification can be obtained if two or more sets of primers are used in successive reactions. In this way, the impact of any non-specific products amplified from non-target areas can be minimised by conducting a further amplification using primers whose binding sites are located internal to those of the first set. The specificity of all the primers is combined, usually leading to a single product. A nested PCR can be performed as a sequential series of separate reactions or it can be performed in a single reaction container. Although the appeal of a single tube nested PCR reaction is obvious, the approach has suffered from problems such as the inconsistency of efficiency between different rounds of amplification, this leading to significant limitations where it has been desired to obtain a quantitative result. In order to effect amplification from different primer sets in a sequential order, many of the prior art methods have largely focussed on the method of lowering the concentration of the outer primers and having a high annealing temperature for the first phase and a lower temperature for the second phase. However, the efficiency of the amplification has differed significantly as between the different primer sets, thereby limiting the utility of this method.

Accordingly, in the context of a nested PCR embodiment of the present invention, the inner primers can be inactivated while the outer primers are undergoing extension. Thereafter, the outer primers can be rendered inactive using antisense oligonucleotides which hybridise to them while inner primer functionality is restored, either by a change in the conditions of the reaction or via a hybridisation between or dissociation of the antisense oligonucleotides and the inner primers. This interaction can occur in any one of a number of ways, which will be discussed in more detail hereafter Hybridisation of the antisense oligonucleotide to the outer and/or inner primers will result in a decrease in concentration of free primers which in turn will lead to inefficiency of hybridisation of that primer to the DNA target template, whereas dissociation will have the opposite effect. Hybridisation is subject to a number of variables. The strength and extent of hybridisation of the antisense oligonucleotide to the primer will depend on its sequence and length, the presence of any mismatches or modifications which either decrease or increase hybridisation, the concentration of the oligonucleotide and the annealing temperature or time. Except for an antisense oligonucleotide for which the most 3′ base of the antisense oligonucleotide hybridises to the most 5′ base of the primer, hybridisation of the antisense oligonucleotide will result in it extending in the 3′ direction. This will produce a longer antisense oligonucleotide which will hybridise more strongly during subsequent cycles.

Reference to an “oligonucleotide” should be understood as a reference to any molecule comprising a sequence of nucleotides, or functional derivatives or analogues thereof. Reference to a “primer” should be understood as a reference to an oligonucleotide which has the function of hybridising to a nucleic acid target and has the actual or potential function of undergoing an extension or ligation reaction. Reference to an “antisense oligonucleotide” should be understood as a reference to an oligonucleotide part or all of which has a complementary sequence to part or all of the oligonucleotide of interest. It should be understood that an oligonucleotide, primer or antisense oligonucleotide may comprise non-nucleic acid components. For example, the oligonucleotide, primer or antisense oligonucleotide may also comprise a non-nucleic acid tag such as a fluorescent or enzymatic tag or some other non-nucleic acid component which facilitates the use of the molecule as a probe or which otherwise facilitates its detection or immobilisation. The oligonucleotide, primer or antisense oligonucleotide may also comprise additional nucleic acid components. In another example, the oligonucleotide, primer or antisense oligonucleotide may be a protein nucleic acid which comprises a peptide backbone exhibiting nucleic acid side chains. Preferably, said primer is a DNA primer.

The antisense oligonucleotide of the present invention is designed to hybridise to an oligonucleotide of interest, such as a primer or probe. Accordingly, by “directed to” is meant that the antisense oligonucleotide hybridises to a region of the subject oligonucleotide of interest. The antisense oligonucleotide may, therefore, hybridise across only part of the oligonucleotide of interest or it may hybridise across the full length of the oligonucleotide of interest. It should be understood that although the antisense oligonucleotide is referred to as an “antisense” oligonucleotide, the use of this terminology is intended to indicate that the nucleotide sequence of this antisense is designed to enable the antisense to hybridise to the subject oligonucleotide of interest, such as a primer. It should also be understood that in terms of the nomenclature used in this specification to describe the antisense oligonucleotide, the end of the antisense oligonucleotide which hybridises to the 3′ end of the oligonucleotide of interest is referred to as the 5′ end of the antisense oligonucleotide while the other end is the 3′ end of the antisense oligonucleotide.

Without limiting the present invention to any one theory or mode of action, the reaction of the present invention may be designed to use either one, or more than one, type of antisense oligonucleotide, depending on the number of oligonucleotides of interest which are present in the reaction and the function of which it is designed to modulate. It should also be understood that in the context of nested amplification reactions, the antisense oligonucleotides which are utilised may be directed towards either one or more forward primers or one or more reverse primers. In another alternative, the reaction may be designed to use both antisense oligonucleotides directed to one or more forward primers and antisense oligonucleotides directed to one or more reverse primers.

In one aspect, the method of the present invention is based on using an antisense:oligonucleotide of interest hybridisation event as the basis to effect a sequence change in either one of these molecules or a further oligonucleotide molecule associated with this complex such that the functionality of the oligonucleotide of interest is ultimately modulated. Reference to “modulate” or “modulation” is intended to mean either that an oligonucleotide of interest which is capable of hybridisation or binding to a target molecule and/or undergoing nucleotide extension is rendered non-functional or that the converse occurs, that is that an oligonucleotide of interest which is inactive in that it cannot undergo hybridisation or binding to a target molecule and/or extension, is rendered functional. In the latter situation, the oligonucleotide of interest may have been rendered inactive due to application of the method of the present invention or it may have been initially produced in this form and thereafter at the appropriate time it can be rendered functional. To this end, reference to “nucleotide sequence change” should be understood as a reference to any change to the nucleotide sequence of an oligonucleotide relative to the sequence prior to the change event and includes, but is not limited to, nucleotide extension of the oligonucleotide of interest, antisense molecule or other oligonucleotide forming part of an oligonucleotide complex, mutation or addition or deletion of one or more nucleotides or degradation of the nucleic acid molecule.

Methods for achieving this nucleotide sequence change include but are not limited to the following:

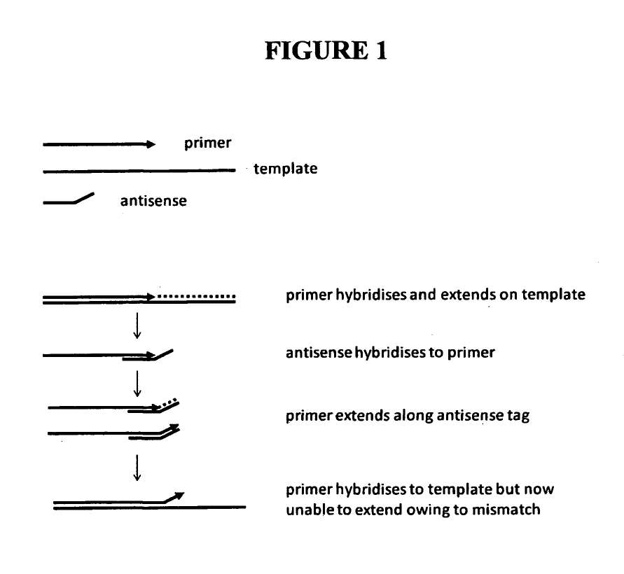

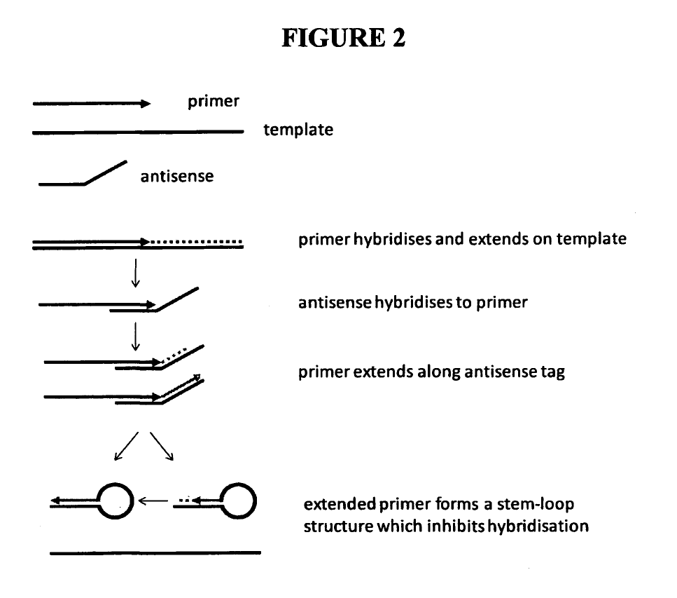

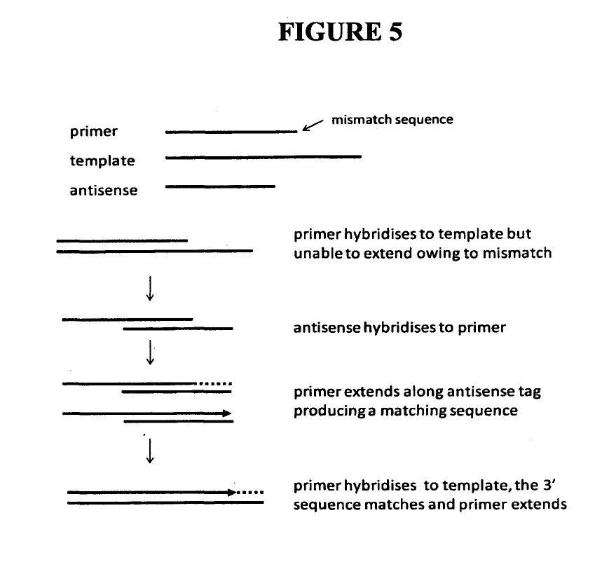

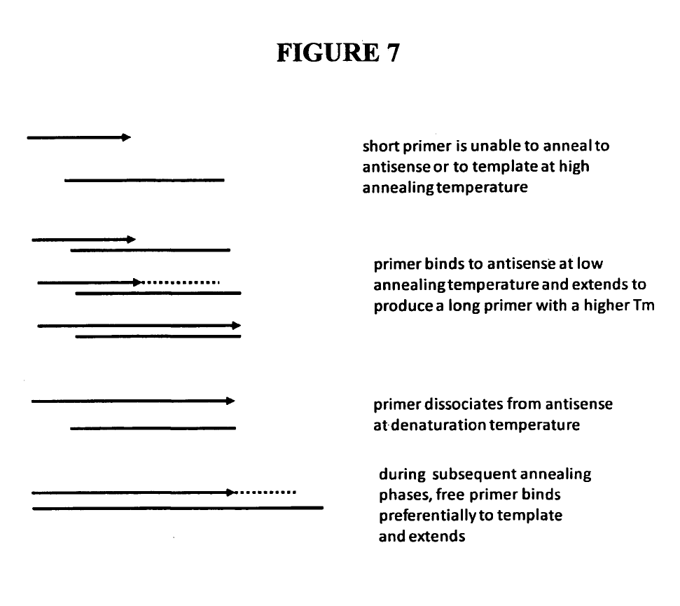

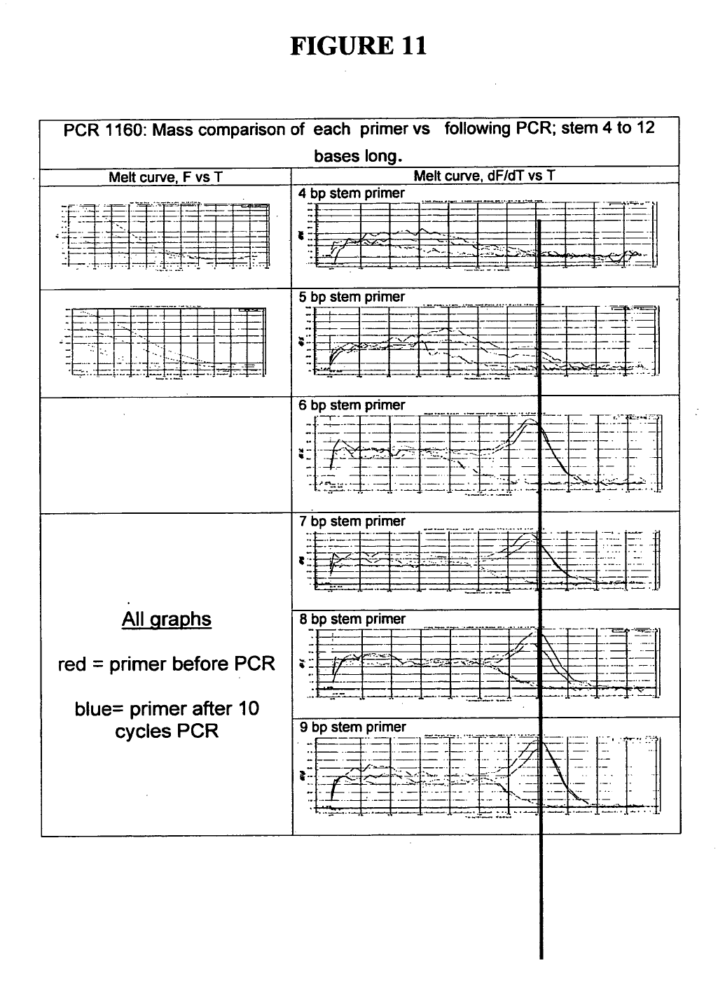

The antisense oligonucleotides in one aspect of the present invention function on the basis of providing a template against which a hybridised functional oligonucleotide of interest, such as a primer, extends and is thereby rendered non-functional or the reverse occurs. This is more specifically described as follows:
(i) 5′ tag on the antisense oligonucleotide.
   This mechanism for inhibiting extension of a primer is based on designing the antisense oligonucleotide to hybridise to the 3′ end of a primer and locating an oligonucleotide tag at the 5′ end of the antisense oligonucleotide (also herein interchangeably referred to as an oligonucleotide extension). A schematic diagram of this mechanism is shown in FIG. 1.
   When the primer hybridises to the tagged antisense oligonucleotide, the primer extends in the 3′ direction. Following dissociation of this duplex the extended primer may hybridise to the complementary template strand. For efficient extension along the template, the sequence of the primer extension must be complementary to that sequence of the template strand which hybridises to the extension, and for this to be the case the sequence of the oligonucleotide tag must be the same as that sequence of the template strand which hybridises to the extension. However, if the sequence of the oligonucleotide tag differs from that sequence of the template strand which hybridises to the extension, further extension and amplification will not take place ie. the primer will have been inactivated. As a consequence, there occurs progressive inactivation of primer molecules with increasing cycle number and this phenomenon synergises with the direct inhibitory effect produced by hybridisation of the antisense oligonucleotide to the outer primer.
   Those skilled in the art will appreciate that the essential property of the tag is to act as a template for 3′ extension of the primer such that the extended primer is unable to act during subsequent amplification. This can be achieved by comprising the 5′ antisense oligonucleotide tag of either normal nucleotides or modified nucleotides, such as either iso-deoxycytosine or iso-deoxyguanine (the complementary nucleotide must be present in the reaction). The greater the difference between the sequence of the oligonucleotide tag and the sequence of the template strand which would hybridise to the extension, the greater the degree of primer inactivation. Particularly efficient inactivation is produced by making the sequence of the oligonucleotide tag exactly complementary to that sequence of the template strand which would hybridise to the extension. Accordingly, as the amplification reaction progresses, either with repetitive annealing during thermal cycling or with annealing during the constant temperature of an isothermal reaction, some molecules of the antisense oligonucleotide will hybridize to the primer. The hybridized primer molecules will extend in the 3′ direction along the tag, the sequence of which has been chosen so that the 3′ end of the extended primer is now mismatched to the corresponding sequence of the template. Thus extended primer molecules, although they can still hybridize to the nucleic acid template cannot extend. Thus as the reaction proceeds the primer molecules are progressively inactivated and are unable to lead to amplification.
   Reference to “oligonucleotide tag” should be understood as a reference to a nucleotide sequence which is linked to the antisense oligonucleotide of the present invention. In one embodiment, the tag is 1-10 bases in length, preferably 2-5 bases in length and more preferably 2-3 bases in length.
   The subject tag is designed such that the nucleotide sequence which is complementary to the sequence of the tag is “mismatched” relative to the nucleotide sequence of the DNA region 5′ of the hybridisation site of the 3′ end of the primer. By “mismatched” is meant that the sequence of the tag is such that subsequently to hybridisation of the primer to the antisense oligonucleotide and the extension of the primer along the tag, only the section of the extended primer which corresponds to the original primer will be able to hybridise to the DNA region of interest and the extended section will be of a sequence which does not facilitate its hybridisation to the DNA region of interest. In this way, any further extension of this primer during amplification is inhibited since the 3′ end of this primer cannot hybridise to the DNA region of interest. Accordingly, when the primer hybridises to the antisense oligonucleotide, the primer extends in the 3′ direction and produces a terminal sequence which prevents efficient extension in the 3′ direction when the primer modified in this way subsequently hybridises to its amplicon template.
   During the course of the reaction, either due to a change in annealing temperature or to the course of time during a constant annealing temperature, the antisense oligonucleotide and the primer hybridise, the primer extends to generate a mismatch to its template which prevents extension.
(ii) Inactivation of a primer owing to the formation of a stem-loop
   A schematic diagram of this mechanism is shown in FIG. 2. The sequence of the 5′ tag of the antisense oligonucleotide is chosen such that, when the primer extends along it, the sequence of the 3′ end of the extended primer becomes complementary to another sequence of the same primer. In this situation, the 3′ end of the extended primer will be able to loop back and hybridise to its complementary sequence. If the complementary sequence does not extend to the 5′ end of the primer, then the 3′ end of the primer will be able to extend along the primer until the 5′ end of the primer is reached. In either case, a stem-loop structure will be produced and, depending upon the length and sequence of the stem, which determines the strength of hybridisation between the 2 arms of the stem, the ability of the stem-loop structure to unfold and for the primer to hybridise to its template will be inhibited. An example of how the 3′ region of an extended primer may hybridise to a 5′ region of the primer and then extend to form a longer stem of a stem-loop ids shown in FIG. 11.
   Modification of the primer in this way may be produced during the reaction by a change in annealing temperature or may occur during the course of time with a constant annealing temperature. Inactivation of the primer by production of a stem loop structure may be of particular value when it is desired to inactivate the primer to prevent it hybridising to its template or to other regions of the genome. This may be useful when the binding site for the inner primer partly overlaps the binding sites for the outer primer since in this circumstance any hybridisation of the outer primer, even if it is inactive by being unable to extend, may inhibit hybridisation of the inner primer. When the 5′ tag of the antisense oligonucleotide is designed to produce stem-loop formation of the extended primer, in nearly all cases the sequence of the extended primer will be such that, even if the stem-loop unfolds and the extended primer binds to its template, there will still be a mismatch and the extended primer will be inactive.
(iii) Re-functionalisation of tagged primer
   A schematic diagram of this mechanism is shown in FIG. 5.
   As would be appreciated, the primer described in point (i) above can be rendered non-functional by virtue of the extension of the primer to incorporate a nucleotide sequence which is mismatched relative to part of the primer hybridisation site. Such a primer, whether generated according to the method described in point (i) or otherwise synthetically or recombinantly produced, can be reactivated using an antisense oligonucleotide which hybridises to the 3′ end of the primer but comprises an additional oligonucleotide tag, the sequence of which corresponds to the sequence of the nucleic acid region of interest. By facilitating extension of the primer along this tag, an extended primer molecule is generated which comprises regions of sequence at its 5′ and 3′ ends which are complementary to the nucleic acid region of interest. The intervening mismatched sequence is sufficiently short, relative to the complementary regions, thereby enabling hybridisation of the primer to the DNA region of interest and subsequent extension, despite the intervening mismatched sequence.
   Accordingly, the primer is initially inactive owing to the presence of two or more nucleotides at its 3′ end which are mismatched to the template. When the antisense oligonucleotide and primer hybridise, the primer extends along the oligonucleotide tag, the sequence of which is such that the sequence of the extension of the primer is now a match to the template.
(iv) Activation of primer via Tm modulation
   Another variation of the activation of a primer by extension along an antisense oligonucleotide can be achieved via modulation of the Tm. A schematic diagram of this mechanism is shown in FIG. 7. The Tm of the native primer is designed such that it hybridises inefficiently to both its template and to the antisense oligonucleotide. However gradually, with the passage of time, primer molecules hybridise to the antisense oligonucleotide, extend and become efficient primers owing to the increase in their Tm. Although hybridisation to the antisense oligonucleotide competes with hybridisation to the template, the 5′ overhang of the primer, which is matched to the template, ensures that hybridisation to the template is favoured.

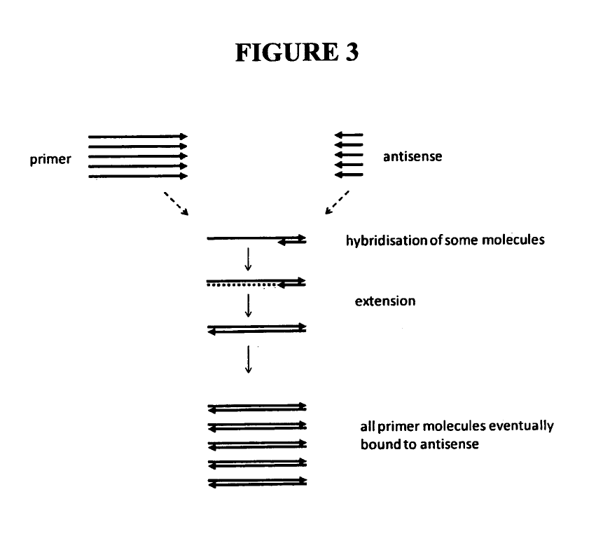

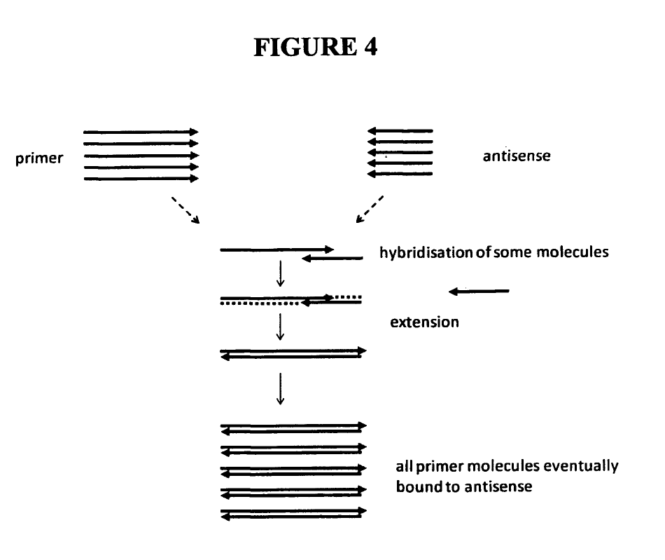

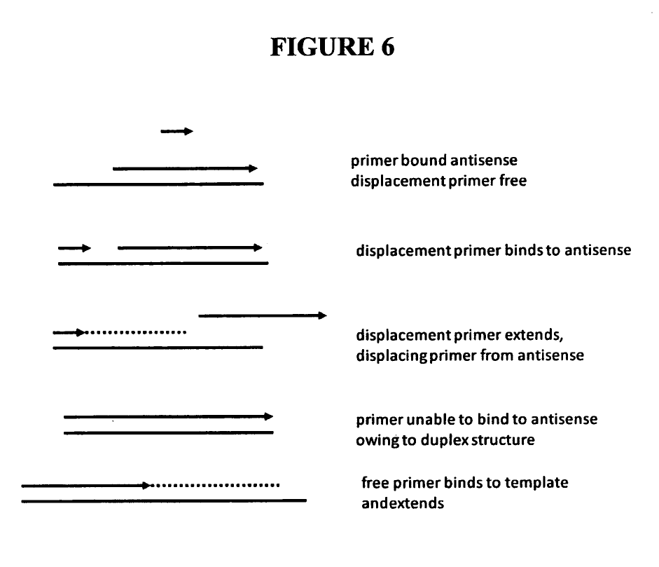

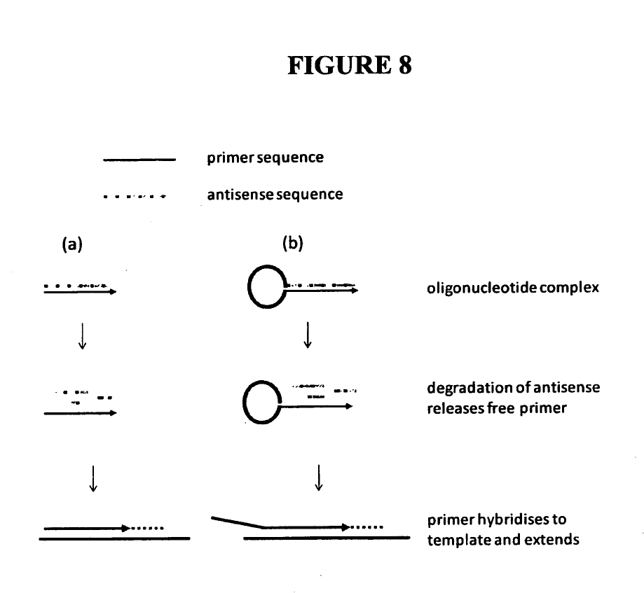

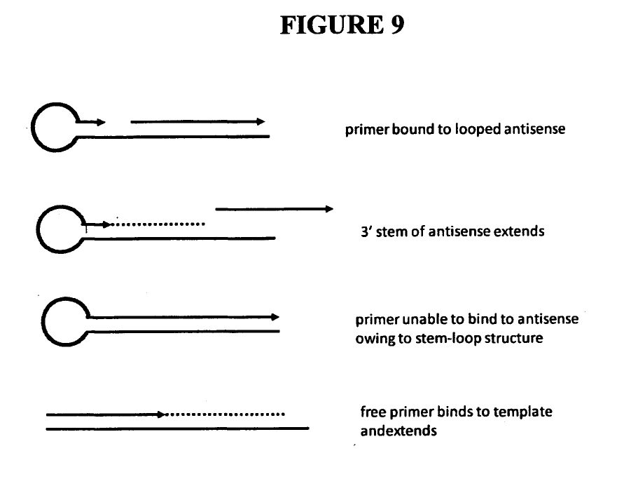

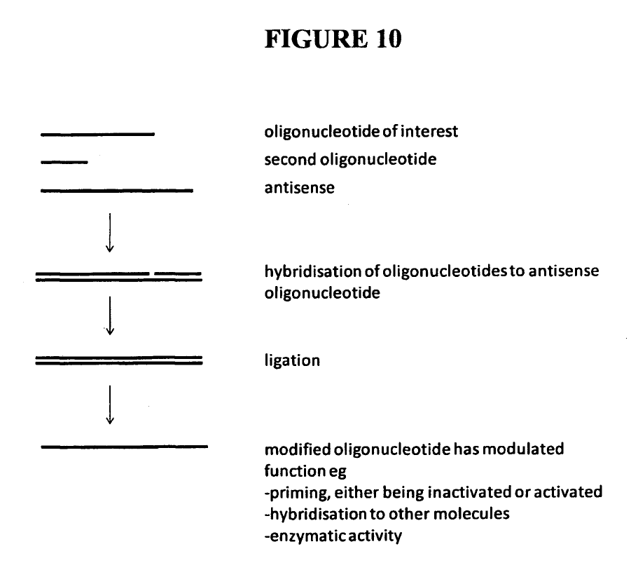

In relation to the second aspect of the invention, the regulation of functionality of an oligonucleotide of interest is based on extension or degradation of the antisense molecule itself. Essentially, this method can be used to either increase or decrease the concentration of free oligonucleotide of interest which can function as a probe or primer. Specifically:
(i) Use of an antisense oligonucleotide which hybridizes to the 3′ end of the oligonucleotide of interest.
   As the amplification reaction progresses, either with repetitive annealing during the thermal cycles or with annealing during the constant temperature of an isothermal reaction, some molecules of the antisense oligonucleotide will hybridize to the oligonucleotide of interest and extend in the 3′ direction along the oligonucleotide of interest. A schematic diagram of this mechanism is shown in FIG. 3. These extended antisense oligonucleotides will hybridize more strongly to the oligonucleotide of interest and their number will progressively increase with time. As a result, increasing inhibition of the oligonucleotide of interest will result and the oligonucleotide of interest activity will progressively “switch off” owing to the decline in the number of free oligonucleotides of interest.
   A variation to this method, a schematic diagram of which is shown in FIG. 4, involves the use of oligonucleotides of interest and antisense oligonucleotide structures which incorporate an additional 5′ tag leading to extension of both the antisense oligonucleotide and the oligonucleotide of interest and producing a duplex which is more tightly hybridised. The sequence of the 5′ tag may be such that the extended oligonucleotide of interest is mismatched to its template in order to ensure that the oligonucleotide of interest extension does not improve binding of the primer to the template.
(ii) Oligonucleotide complex
   In yet another embodiment, the antisense molecule may form part of an oligonucleotide complex. By “oligonucleotide complex” is meant a complex which comprises two or more associated oligonucleotides. It should be understood that in the context of the present invention, additional oligonucleotides may become associated with the complex or, alternatively, one or more oligonucleotides which form part of the complex may become dissociated or degraded. It should also be understood that the subject “complex” includes reference to separate oligonucleotides which are associated via hybridisation or oligonucleotides which are joined, via, for example, by phosphodiester or other chemical bonds, to form a single sequence which can then form a stem loop structure. This complex therefore comprises a single DNA strand which has functionally distinct' regions. For example, the complex may take the form of a stem-loop structure wherein at one end of this structure is the oligonucleotide of interest while at the other end is the antisense sequence. Where the antisense end of this structure folds back on itself to hybridise with the oligonucleotide of interest end, antisense “hybridisation” has occurred. Accordingly, it should be understood that the “antisense oligonucleotide” may be a separate molecule to the oligonucleotide of interest or it may be attached to or otherwise continuous with the oligonucleotide of interest, as described above.
   In one example, a schematic diagram of which is shown in FIG. 6, activation of an oligonucleotide of interest may be achieved using a displacement primer. The oligonucleotide complex initially comprises an oligonucleotide of interest, such as a primer molecule, hybridised to an antisense molecule. The oligonucleotide of interest is therefore initially inactive owing to being hybridised to the antisense molecule. When a displacement primer is introduced to the complex, it hybridises to the antisense molecule and undergoes extension, thereby displacing the oligonucleotide of interest. The free oligonucleotide of interest is thereby rendered functional. To avoid degradation of the oligonucleotide of interest, the polymerase mediating extension of the displacement primer must have displacing activity but not 5′-3′ nuclease activity.
   In another example, a schematic diagram of which is shown in FIG. 8(a), activation of an oligonucleotide of interest may be achieved by degradation of the antisense oligonucleotide. The oligonucleotide complex initially comprises an oligonucleotide of interest, such as a primer molecule, hybridised to a potentially degradable antisense molecule. The oligonucleotide of interest is therefore initially inactive owing to being hybridised to the antisense molecule. The antisense sequence may comprise one or a number of ribonucleotides or modified ribonucleotides. If so, then the rate of hydrolysis of dinucleotide bonds will be increased. The ribonucleotide rate of hydrolysis can be further increased by incorporating into the reaction a ribonuclease such as RNase A or RNase H together with a divalent cation, or incorporating into the reaction a cation of a lanthanide such as terbium. Progressive hydrolysis of the antisense sequence will free the oligonucleotide of interest and its activity will gradually increase.
   The features of a stem-loop oligonucleotide complex may include, but are not limited to:
      (a) An oligonucleotide of interest and an oligonucleotide which is designed to have a stem-loop configuration with the 3′ strand of the stem hybridising to the 5′ strand but where the 5′ strand is longer than the 3′ strand, and comprises a single stranded region which is complementary to the oligonucleotide of interest and thereby capable of hybridising to and inactivating any free oligonucleotide of interest molecules. Accordingly, the 5′ strand of the stem is the antisense sequence and renders the oligonucleotide of interest molecules non-functional. A schematic diagram of this mechanism is shown in FIG. 9.
         However this is a dynamic situation, with the stem opening and closing and the oligonucleotide of interest binding and unbinding. Consequently, the amplification cycles can be designed to facilitate 3′ extension of the stem-loop molecule. With each cycle the antisense molecule will be extended and develop a large stem which will not open up in subsequent cycles. Oligonucleotide of interest molecules will therefore be unable to hybridise and will be able to bind to their DNA region of interest. Again it is desirable that the polymerase mediating 3′ extension of the stem should not have 5′-3′ nuclease activity.
         Where there is utilised an antisense oligonucleotide which comprises a stem loop structure with a secondary antisense sequence, the length and Tm of the mini-stem are chosen so that as the reaction progresses, there occurs 3′ extension of the secondary antisense sequence, so that the overall antisense oligonucleotide forms a definitive stem-loop structure with an extended stem. Antisense activity is thus progressively lost and oligonucleotide of interest activity progressively increases.
      (b) An oligonucleotide which is designed to have a stem-loop configuration with the 3′ strand of the stem having the oligonucleotide of interest sequence and the 5′ strand of the stem having the antisense sequence will be unable to act as a functional oligonucleotide, since the stem-loop configuration is far more energetically favourable than the unfolded configuration, which is required for effective functionality. However, although the antisense oligonucleotide and the oligonucleotide of interest are part of a single stem-loop molecule, the antisense moiety of the stem is potentially degradable. Initially, the oligonucleotide of interest sequence is strongly hybridised to the antisense sequence, owing to the stem-loop structure. A schematic diagram of this mechanism is shown in FIG. 8(b). With the passage of time, degradation of the antisense sequence occurs progressively and, as the result of the disappearance of the stem-loop structure, the oligonucleotide of interest sequence is now free and able to hybridise to its template. One mechanism of degradation is the presence within the antisense of one or more RNA nucleotides and, if necessary, the presence of a ribonuclease in the reaction mixture.
         The antisense sequence may comprise one or a number of ribonucleotides or modified ribonucleotides. If so, then the rate of hydrolysis of dinucleotide bonds will be increased. The ribonucleotide rate of hydrolysis can be further increased incorporating into the reaction a ribonuclease such as RNase A or RNase H together with a divalent cation. Progressive hydrolysis of the antisense sequence will result in dissociation of a linear antisense oligonucleotide from the oligonucleotide of interest or unfolding of the stem-loop and loss of antisense inhibition, so that oligonucleotide of interest activity will gradually increase.
      (c) In yet another example of an oligonucleotide complex, a schematic diagram of which is shown in FIG. 10, the complex will be formed by an antisense oligonucleotide which binds to the 3′ end of the oligonucleotide of interest and which has a 5′ extension which provides a binding site for a second oligonucleotide which hybridises immediately 3′ to the oligonucleotide of interest. Under the action of a ligase enzyme, the oligonucleotide of interest becomes ligated to said second oligonucleotide forming a sequence which results in loss or gain of function of said oligonucleotide of interest and said function involves interaction with a target molecule. During the course of the reaction, hybridisation and ligation of the second oligonucleotide may occur progressively during the reaction or may be controlled by controlling the conditions of the reaction.

Accordingly, one aspect of the present invention is directed to a method of modulating the capacity of an oligonucleotide of interest to undergo extension along a target nucleic acid, said method comprising hybridising said oligonucleotide to the 3′ end of an antisense oligonucleotide and facilitating the 3′ extension of said oligonucleotide of interest along said antisense oligonucleotide wherein the antisense nucleotide sequence along which said oligonucleotide of interest extends generates an extension of said oligonucleotide which is either:
(i) complementary to the nucleotide sequence of the target nucleic acid and thereby renders said oligonucleotide of interest functional;
(ii) mismatched relative to the nucleotide sequence of the target nucleic acid and thereby renders said oligonucleotide of interest non-functional; or
(iii) complementary to a nucleotide sequence of another region of said oligonucleotide of interest and wherein said extension hybridises to said region of said oligonucleotide of interest thereby forming a stem-loop configuration and rendering said oligonucleotide of interest non-functional.

In one embodiment, said oligonucleotide of interest is a primer.

In another aspect there is provided a method of modulating the functionality of an oligonucleotide of interest, said method comprising:
(i) hybridising an antisense oligonucleotide to the 3′ end of said oligonucleotide of interest and facilitating the 3′ extension of said antisense oligonucleotide along said oligonucleotide of interest thereby rendering said oligonucleotide of interest non-functional; or
(ii) hybridising the 3′ end of an antisense oligonucleotide to the 3′ end of said oligonucleotide of interest and facilitating the 3′ extension of said antisense oligonucleotide along said oligonucleotide of interest and the 3′ extension of said oligonucleotide of interest along said antisense oligonucleotide thereby rendering said oligonucleotide of interest non-functional; or
(iii) contacting an oligonucleotide complex, which oligonucleotide complex comprises an oligonucleotide of interest hybridised to an antisense oligonucleotide, with a primer which hybridises to said antisense oligonucleotide at a region which is 3′ to the region to which the oligonucleotide of interest is hybridised and effecting 3′ extension of said primer wherein extending said primer displaces said oligonucleotide of interest and renders said oligonucleotide of interest functional; or
(iv) extending the linear 3′ strand of a looped antisense oligonucleotide, which 3′ linear strand is hybridised to a 5′ linear strand but which 5′ linear strand is longer than the 3′ linear strand and comprises a single stranded region which is complementary to said oligonucleotide of interest and which is hybridised to said oligonucleotide of interest, wherein extending said 3′ linear strand along said single stranded region displaces said oligonucleotide of interest and renders said oligonucleotide of interest functional; or
(v) selectively degrading the linear antisense strand either of a stem-loop nucleic acid molecule or of a duplex between said linear antisense strand and an oligonucleotide of interest, which degradation renders the complementary linear oligonucleotide of interest strand single-stranded and thereby functional; or
(vi) hybridising oligonucleotide of interest with a low Tm to an antisense oligonucleotide and effecting extension of said oligonucleotide of interest wherein said extended oligonucleotide of interest develops an increased Tm and is thereby rendered functional; or
(vii) contacting an oligonucleotide complex, which oligonucleotide complex comprises an oligonucleotide of interest hybridised to the 3′ end of an antisense oligonucleotide and a second oligonucleotide hybridised to said antisense oligonucleotide 3′ to the region to which the oligonucleotide of interest is hybridised and effecting ligation between said oligonucleotide of interest and said second oligonucleotide wherein the functionality of said oligonucleotide of interest is modulated.

In one embodiment, said nucleic acid is DNA.

In another embodiment said oligonucleotide of interest is directed to a target molecule which is either a protein or a nucleic acid molecule.

In another embodiment functionality of said oligonucleotide of interest is functionality as a primer, aptamer, DNAzyme, RNase or ribozyme.

Accordingly, there is provided a method of modulating the capacity of a primer to undergo extension along a target DNA, said method comprising hybridising said primer to the 3′ end of an antisense oligonucleotide and facilitating the 3′ extension of said primer along said antisense oligonucleotide wherein the antisense nucleotide sequence along which said primer extends generates a primer extension which is either:
(i) complementary to the nucleotide sequence of the target DNA and thereby renders said primer functional; or
(ii) mismatched relative to the nucleotide sequence of the target DNA and thereby renders said primer non-functional; or
(iii) complementary to a nucleotide sequence of another region of said primer and wherein said extension hybridises to said region of the primer thereby forming a stem-loop configuration rendering said primer non-functional.

In another aspect there is provided a method of modulating the functionality of a DNA primer said method comprising:
(i) hybridising an antisense oligonucleotide to the 3′ end of said primer and facilitating the 3′ extension of said antisense oligonucleotide along said primer thereby rendering said primer non-functional; or
(ii) hybridising the 3′ end of an antisense oligonucleotide to the 3′ end of said primer and facilitating the 3′ extension of said antisense oligonucleotide along said primer and the 3′ extension of said primer along said antisense oligonucleotide thereby rendering said primer non-functional; or
(iii) contacting an oligonucleotide complex, which oligonucleotide complex comprises a first primer hybridised to an antisense oligonucleotide, with a second primer which hybridises to said antisense oligonucleotide at a region which is 3′ to the region to which the first primer is hybridised and effecting 3′ extension of said second primer wherein extending said second primer displaces said first primer and renders said first primer functional; or
(iv) extending the linear 3′ strand of a looped antisense oligonucleotide, which 3′ linear strand is hybridised to a 5′ linear strand but which 5′ linear strand is longer than the 3′ linear strand and comprises a single stranded region which is complementary to said primer and which is hybridised to said primer, wherein extending said 3′ linear strand along said single stranded region displaces said primer and renders said primer functional; or
(v) selectively degrading the linear antisense strand either of a stem-loop nucleic acid molecule or of a duplex between said linear antisense strand and an oligonucleotide of interest, which degradation renders the complementary linear primer strand single-stranded and thereby functional; or
(vi) hybridising a primer with a low Tm to an antisense oligonucleotide and effecting extension of said primer wherein said extended primer develops an increased Tm and is thereby rendered functional; or
(vii) contacting an oligonucleotide complex, which oligonucleotide complex comprises a primer hybridised to the 3′ end of an antisense oligonucleotide and an oligonucleotide hybridised to said antisense oligonucleotide 3′ to the region to which the primer is hybridised and effecting ligation between said primer and said oligonucleotide wherein the functionality of said primer is modulated.

It should be understood that the method of the present invention may be designed such that all the antisense oligonucleotides are of the same type of structure or it may be designed such that any two or more antisense oligonucleotide structure types are used in the one method.

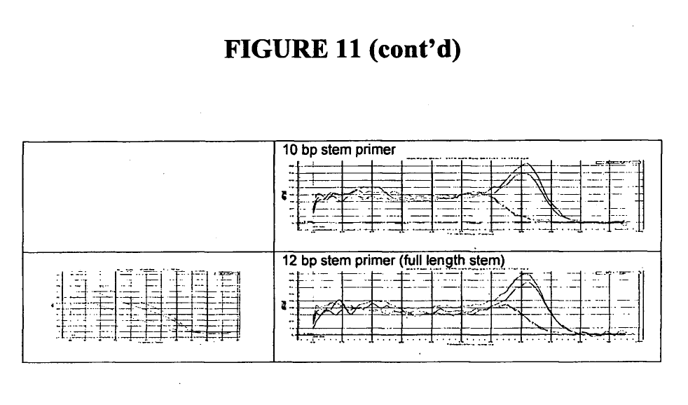

As detailed hereinbefore, there is a wide range of applications for the methods of the present invention including, but not limited to:
(i) Inactivating methods used during nucleic acid amplification. This can be applied in the context of amplification. Uses include, but are not limited to:
   Nested amplification.
   Inactivation can be designed to involve the outer primers and the reaction would be performed under conditions which would ensure that the inner primers were active once the outer primers had become inactive. Those skilled in the art would know that such conditions would include (i) an annealing temperature sufficient to enable activity of the inner primers throughout the reaction, or (ii) a stepwise or gradual decrease in annealing temperature such that the inner primers were initially inactive but became active during the course of the reaction, or (iii) use of an antisense oligonucleotide, as previously described, to convert one or more inactive inner primers to activity during the course of the reaction. The use of antisense oligonucleotides to enable performance of a nested PCR in a closed single tube is described in Brisco et al. (2011).
   Production of single-stranded DNA.
   Production of single-stranded DNA towards the end of the nucleic acid amplification reaction can be useful for various purposes including sequencing, quantification, and the ability to use a wide range of probes to detect amplified product. The production of single-stranded DNA by amplification reactions involving one or more antisense oligonucleotides can be achieved by adjusting reaction conditions so that one primer is inactivated earlier in time than the other primer. Those skilled in the art will realise that one way of achieving this is to use only one antisense oligonucleotide and to adjust reaction conditions, such as annealing temperature, so that the target primer becomes inactivated after a defined number of cycles, thus allowing the other primer to continue annealing and extending to produce increasing amounts of single-stranded DNA. An example of this use is shown in FIG. 12.
(ii) Activating methods during nucleic acid amplification. This can be applied in the context of either PCR or isothermal amplification and they can be used in the context of nested amplification, For nested amplification ensuring that the inner primers are active only during later cycles of the nucleic acid amplification minimises the opportunity for the inner primers to produce non-specific amplification, either of completely non-specific products or of products which have a close sequence relationship to the target product but for which amplification is not desired.
   Again, conditions can be such that activation occurs gradually during the course of the reaction, at a constant annealing temperature, or the temperature conditions can be adjusted to control the point in the reaction at which activation occurs.
(iii) Both activating and inactivating methods can also be used when the oligonucleotide of interest in its active state functions as an aptamer, which hybridises to a protein molecule and causes a change in structure or function of said protein molecule, or as a DNAzyme or ribozyme, which consist of DNA, or RNA respectively and hybridise to a nucleic acid molecule and act as an enzyme.

Accordingly, in one embodiment the present invention enables the inducible inactivation or activation of the primers which are sought to be used in a nested amplification reaction, thereby enabling the design of a single tube nested PCR method which maintains a constant and optimal level of efficiency. More specifically, this development has enabled a degree of control and robustness not previously available in the context of a single tube nested PCR. The method of the present invention is useful in the context of any application which requires the analysis of a specific DNA region of interest, such as a specific gene. Still further, this method is applicable to improve both thermal and isothermal nucleic acid amplification reactions.

According to this embodiment there is provided a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed; and
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable extension of said first primers due to antisense oligonucleotide hybridisation to said first primers.

Appreciation of the various factors influencing hybridisation will help those skilled in the art to design a single-tube nested PCR involving one or more antisense oligonucleotide primers such that (a) hybridisation of an antisense oligonucleotide to a primer is minimal or absent during the phase where that primer is required to anneal and extend but occurs during the phase when other primers are required to preferentially anneal and extend and/or (b) hybridisation of an antisense oligonucleotide to another primer is minimal or absent during the phase where that primer is required not to be able to anneal and extend and other primers are required to preferentially anneal and extend but occurs during the phase when that primer is required to preferentially anneal and extend.

Reference to “forward primer” should be understood as a reference to a primer which amplifies the target DNA in the DNA sample of interest and in the PCR by hybridising to the antisense strand of the target DNA.

Reference to “reverse primer” should be understood as a reference to a primer which amplifies the target DNA in the DNA sample of interest and in the PCR by hybridising to the sense strand of the target DNA.

Reference to “downstream” or “upstream” in relation to location should be understood as reference to location relative to the sense DNA strand, with “downstream” referring to 3′ and “upstream” referring to 5′.

As detailed hereinbefore, a nested nucleic acid amplification reaction, such as a PCR or isothermal reaction, is predicated on the use of two or more sets of forward and reverse primers which are directed to hybridising to progressively more internal sequences within the target DNA. In the context of the present invention, reference to the “first” forward and reverse primer should be understood as a reference to the primers which hybridise at the outermost positions of the target DNA. The “second” forward and reverse primers should be understood as a reference to internal primers. That is, these second primers are designed to hybridise to a sequence which is downstream of the first forward primer and upstream of the first reverse primer, respectively. It is within the skill of the person in the art to design at what intervals along the target DNA these primers will hybridise. It should be understood that one may also adopt the method to incorporate a third forward and reverse primer set which is designed to hybridise to a sequence which is downstream of the second forward primer and upstream of the second reverse primer, respectively.

Without limiting the present invention to any one theory or mode of action, the reaction of the present invention may be designed to use a semi-nested rather than a nested reaction. In this case, either a second forward or a second reverse primer is not used and antisense oligonucleotides directed to the single forward or single reverse primer that remains are not present.

In one embodiment the present invention is directed to a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable and which antisense oligonucleotide directed to said first primer comprises a 5′ nucleic acid tag sequence, the complementary nucleotide sequence of which tag is mismatched relative to the nucleotide sequence of the DNA region adjacent to the 5′ end of the hybridisation site of said primer; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said first primer along said tagged antisense oligonucleotide.

In another embodiment the present invention is directed to a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable and which antisense oligonucleotide directed to said first primer comprises a 5′ nucleic acid tag sequence, the complementary nucleotide sequence of which tag is complementary relative to the nucleotide sequence of another region of the same primer; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said first primer along said tagged antisense oligonucleotide.

In another embodiment there is provided a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed; and
   (c) one or more antisense oligonucleotides as defined hereinbefore directed to one or more of said primers wherein the functionality of said primers is thereby modulatable and one or more of which antisense oligonucleotides hybridise to the 3′ end of said first primer and are extendible in the 3′ direction along said primer; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said antisense oligonucleotide along said primer.

In one embodiment, said antisense oligonucleotide hybridises to the 3′ end of said primer and extends along said primer.

In another embodiment, the 3′ end of said antisense oligonucleotide hybridises to the 3′ end of said primer and the 3′ end of said antisense oligonucleotide extends along said primer and the 3′ end of said primer extends along said antisense oligonucleotide.

In still another embodiment there is provided a method of amplifying a target DNA, said method comprising:
(i) contacting a DNA sample with:
   (a) a first forward primer directed to said target DNA and a first reverse primer directed to said target DNA; and
   (b) a second forward primer directed to said target DNA wherein said second forward primer is directed to a nucleic acid sequence located downstream to the sequence to which said first forward primer is directed
      a second reverse primer directed to said target DNA wherein said second reverse primer is directed to a nucleic acid sequence located upstream to the sequence to which said first reverse primer is directed
      and which second primers comprise a 3′ tag which is mismatched relative to the nucleotide sequence of the target DNA; and
   (c) one or more antisense oligonucleotides as defined hereinbefore directed to one or more of said primers wherein the functionality of said primer is thereby modulatable and one of said antisense oligonucleotide sequences comprises both a 3′ region complementary to the 3′ region of said second primer and a more 5′ region the complementary sequence of which is complementary relative to the sequence of the target DNA adjacent to the primer binding site; and
   wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which enable hybridisation and extension of said second primers along said antisense oligonucleotide; and
(iv) amplifying the DNA sample of step (iii) under conditions which enable hybridisation and extension of said second primers along the DNA region of interest but which do not enable extension of said first primers due to antisense oligonucleotide hybridisation to said first primers.

The method of the present invention therefore enables an initial amplification off the outermost primers to initially proceed efficiently as the dominant amplification reaction. However, since it is sought to conclude this amplification and to proceed with amplification off the internal second primer set, effecting the hybridisation of the antisense oligonucleotide to the first forward primer results in the generation of primers which are effectively blocked from undergoing any further extension in the context of the target DNA. Together with release of the internal primers from the antisense oligonucleotide to which they are bound, the ongoing unwanted amplification of the outer primers is minimised and the amplification of the inner primers can proceed under conditions which facilitate efficient amplification. It should be understood that one need not necessarily utilise antisense oligonucleotides directed to the inner primers, for example if the outer primers effectively themselves hinder hybridisation of the inner primer to their hybridisation site.

As detailed hereinbefore, the present invention is predicated on the fact that in some amplification situations it may be desirable to have one or a pair of primers active during initial cycles of amplification and to then suddenly or gradually become inactive, and to have one or another pair of primers inactive during the initial cycles and to then suddenly or gradually become active during the later cycles.

Facilitating the interaction of the primer with antisense oligonucleotide and the primer with the target DNA may be performed by any suitable method. Those methods will be known to those skilled in the art. To this end, it should be understood that the antisense oligonucleotide and/or the inner primers can be incorporated into the reaction tube at any suitable time point. While incorporation is generally prior to the commencement of the initial amplification cycles, that is together with the forward and reverse outer primers, incorporation of one or more may be performed subsequently to the initial amplification cycles with the outer primers. The mode of incorporation of the antisense oligonucleotide and/or the inner primers will depend on how the skilled person is seeking to perform the amplification reaction but, in general, for ease of use and avoidance of contamination, it is usually desirable to be able to perform the entire reaction in a single tube. Nevertheless, any other method of achieving the steps of the invention can be used.

Methods for achieving primer directed amplification are also very well known to those of skill in the art. In a preferred method, said amplification is polymerase chain reaction.

Reference to a “sample” should be understood as a reference to either a biological or a non-biological sample. Examples of non-biological samples includes, for example, the nucleic acid products of synthetically produced nucleic acid populations. Reference to a “biological sample” should be understood as a reference to any sample of biological material derived from any living creature such as, but not limited to an animal, plant or microorganism (including cultures of microorganisms) and such as, but not limited to, cellular material, blood, mucus, faeces, urine, tissue biopsy specimens, fluid which has been introduced into the body of an animal and subsequently removed (such as, for example, the saline solution extracted from the lung following lung lavage or the solution retrieved from an enema wash), plant material or plant propagation material such as seeds or flowers or a microorganism colony. The biological sample which is tested according to the method of the present invention may be tested directly or may require some form of treatment prior to testing. For example, a biopsy sample may require homogenisation prior to testing. Further, to the extent that the biological sample is not in liquid form, it may require the addition of a reagent, such as a buffer, to mobilise the sample.

To the extent that the target DNA or other molecule is present in a biological sample, the biological sample may be directly tested or else all or some of the nucleic acid material present in the biological sample may be isolated prior to testing. It is within the scope of the present invention for the target nucleic acid molecule to be pre-treated prior to testing, for example inactivation of live virus or being run on a gel. It should also be understood that the biological sample may be freshly harvested or it may have been stored (for example by freezing) prior to testing or otherwise treated prior testing (such as by undergoing culturing).

Reference to “contacting” the sample with the primer or antisense oligonucleotide should be understood as a reference to facilitating the mixing of the primer with the sample such that interaction (for example, hybridisation) can occur. Means of achieving this objective would be well known to those of skill in the art. Without limiting the present invention to any one theory or mode of action, the extent of contact is determined by the reaction conditions as well as the respective Tm values of the primer and the antisense oligonucleotide. Contact might be manipulated by controlling annealing temperature, so that contact does not occur at a high temperature but occurs at a lower temperature. Or contact might be allowed to occur at a constant rate during a constant temperature. Or extent of contact might be changed progressively owing to progressive change in annealing temperature such as during a touchdown PCR.

The choice of what type of sample is most suitable for testing in accordance with the method disclosed herein will be dependent on the nature of the situation, such as the nature of the condition being monitored. For example, in a preferred embodiment a neoplastic condition is the subject of analysis. If the neoplastic condition is a leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one mammal. In another example, in another preferred embodiment an infectious disease condition is the subject of analysis. In order to determine the nature and site of infection, samples of blood, bodily fluid, excretion or tissue would be likely to provide a suitable sample for testing and the primers necessary for amplification of particular nucleic acid sequences would be chosen so that detection would be directed against the range of organisms likely to be responsible for the infection. Choosing an appropriate sample and amplification system for any given detection scenario would fall within the skills of the person of ordinary skill in the art.

The term “mammal” to the extent that it is used herein includes humans, primates, livestock animals (e.g. horses, cattle, sheep, pigs, donkeys), laboratory test animals (e.g. mice, rats, rabbits, guinea pigs), companion animals (eg. dogs, cats) and captive wild animals (eg. kangaroos, deer, foxes). Preferably, the mammal is a human or a laboratory test animal. Even more preferably the mammal is a human. The term “organism” to the extent that it is used herein refers to any living entity, including but not limited to animals, plants, bacteria, viruses and fungi.

The present invention is further described by reference to the following non-limiting examples.

### EXAMPLE 1

Table 1 shows the results of 2 experiments illustrating conversion by antisense of the active outer primer to an inactive state and of the inactive inner primer to an active state.

Tables 2, 3, and 4 show various aspects of the protocol used.

### EXAMPLE 2

### Nucleic Acid Amplification During Thermal Cycling

The following method involves the use of antisense oligonucleotides to switch off outer primers and switch on inner primers during the course of a PCR. Conditions are adjusted so that the inner primers are switched on to full functionality before the point at which functionality of the outer primers has been lost.

Method

PCR: Use a 25 μL reaction containing 100 ng of each outer primer, 100 ng of each inner primer, each antisense oligonucleotide at a molar concentration equal to that of the corresponding outer and inner primer, 2.5 mM magnesium, 200 nM dATP, dTTP, dGTP and dCTP, 1 u Platinum Taq (Invitrogen), a Taqman hydrolysis probe and various masses of DNA in a reaction containing 20 mM Tris-HCl pH 8.4, 50 mM KCl, 2.5 mM MgCl₂. The cycling conditions are: 96° C. for 2 minutes then 40 cycles of 94° C. for 15 seconds, 58° C. for 60 seconds, 72° C. for 60 seconds.

Outer and inner Primers: To calculate Tm values use a base stacking model (based on Borer P. N. et al. (1974), J. Mol. Biol. 86:843 & SantaLucia, J. (1998) Proc. Nat. Acad. Sci. USA 95:1460, available at http://www.promega.com/biomath/calc11.htm.), and assuming 2.5 mM magnesium, 200 nM dATP, dTTP,dGTP and dCTP and 100 ng of primer. Use a gradient of annealing temperature to determine the 3ge of temperatures over which the primers produce efficient amplification. Outer and inner primers with a Tm of 60-65° C., are satisfactory.

The inner primers are designed so that the two bases at the 3′ end produce a mismatch when the primers hybridise to the template strand. This results in the primers being inactive during the initial cycles of the PCR.

Antisense oligonucleotides:
(a) Those to the outer primers are based on the antisense sequence to the 3′ end of the outer primers and carry 5′ and 3′ tags, each of 3 nucleotides.
   Since the outer primer would extend along the 5′ tag after hybridisation, the 3′-5′ sequence of the tag is complementary to the 3′-5′ sequence of the template strand immediately downstream of the point at which the native primer would hybridise. This design results in a 3 base mismatch between the 3′ end of the extended primer and its template, which blocks the outer primer from extending further, and thus prevents amplification of the target. The sequence of the 3 bases at the 3′ end of the oligonucleotide is such that each potentially hybridising base of the oligonucleotide is the same as the potentially hybridising base of the primer. Again, this design results in a 3 base mismatch, which blocks extension of the oligonucleotide. This strategy prevents production of an unduly long antisense oligonucleotide which has a higher Tm and which might exert unwanted inhibition during the initial high-temperature phase of the PCR. Extension of the antisense oligonucleotide in the 3′ direction can also be prevented by an amine modification at the 3′ end.
(b) antisense oligonucleotides to the inner primers are based on the antisense sequence to the 3′ end of the inner primers, including the mismatch bases. They carry a 5′ tag and either a tag or an amine modification at the 3′ end. The 5′.tag comprises 4-6 bases, with the sequence being chosen so that when hybridisation to the primer occurs, the primer extends producing a 3′ sequence which perfectly matches the template sequence.

For the antisense oligonucleotides binding to the outer primers, a Tm range of 48-55° C. for the core hybridising sequence of the oligonucleotide, results in satisfactory progressive inhibition of the outer primers. For the antisense oligonucleotides binding to the inner primers, a Tm range of 52-56° C. produces satisfactory progressive activation of the inner primers. Generally, Tm values for the antisense oligonucleotides directed against the outer primers should be 49-51° C., whereas those directed against the inner primers should be 52-54° C.

### EXAMPLE 3

### Isothermal Nucleic Acid Amplification

The following protocol uses antisense oligonucleotides to sequentially switch off the activity of one set of primers and switch on the activity of another set of primers during isothermal nucleic acid amplification. The primers of the second set are targeted to sites internal to the sites targeted by the first set of primers, ie., the reaction is a nested amplification. Helicase dependent amplification (HDA) is the method used for isothermal amplification.

Method

HDA involves use of the IsoAmp II Universal tHDA kit supplied by Biohelix Corporation. (http://www.biohelix.com/default.asp). The detailed amplification protocol and instructions for HDA are provided at http://www.biohelix.com/pdf/-H0110S_full_version_BH.pdf. and protocol C is the protocol generally used.

Use of antisense oligonucleotides involves the following modifications and/or additions to the reaction:

100 ng each of all 4 primers.

antisense oligonucleotides—each is present at a concentration which is equimolar to that of its target primer. The oligonucleotides directed to the outer primers have Tms of approximately 50° C. and those directed to the inner primers have Tms of approximately 53° C. incubation −60° C. for 120 minutes.

### EXAMPLE 4

### Extension of Stem of Stem-Loop Primer During PCR

The results of the experiment are shown in FIG. 11. The experiment illustrates portion of the strategy illustrated in FIG. 2. Primers with a 3′ stem of 4-12 bases, with a sequence which potentially enabled the stem to loop back and hybridise to a more 5′ region of the primer, were present in a PCR reaction for 10 cycles. Potential extension of the stem was then analysed by performing melting curves. The data in the figure indicate that 4 or 5 base stems were not extended by Taq polymerase whereas stems of 6 or more bases were fully extended to a length of 12 bases.

### EXAMPLE 5

### Primer Inactivation to Produce Single-Stranded DNA

Single-stranded DNA has a number of uses. In this experiment, shown in FIG. 12, an antisense oligonucleotide directed against one primer was incorporated into the PCR. The first 15 cycles were performed with an annealing temperature of 72° C. and exponential amplification occurred during these cycles. The annealing temperature was then decreased to 58° C., gradual inactivation of one primer by its antisense oligonucleotide then developed and production of single-stranded DNA resulting from extension of the other primer then ensued. Two different antisense oligonucleotides, one with a 12 base core and the other with a 13 base core, were used.

### EXAMPLE 6

### Antisense PCR: a Simple and Robust Method for Performing Nested Single-Tube PCR

Materials and Methods

PCR

Unless otherwise stated, a 25-μl reaction contained 100 ng of each outer primer, 200 ng of each inner primer, each antisense oligonucleotide at a molar concentration equal to that of the corresponding outer primer, 2.5 mM magnesium, 200 nM each of dATP, dTTP, dGTP, and dCTP, 1 U of Platinum Taq (Invitrogen), a Taqman hydrolysis probe, and 30 pg to 100 ng of DNA. The reactions were set up manually, and PCR was performed in 20 mM Tris-HCl (pH 8.4), 50 mM KCl, and 2.5 mM MgCl₂. Unless otherwise stated, the cycling conditions were as follows: hot start, 96° C. for 2 min; 15 cycles of a high-temperature “first phase”, 94° C. for 15 s and 72° C. for 60 s; incubation for 5 min at 58° C.; and then 40 cycles of a low-temperature “second phase”, 94° C. for 15 s, 58° C. for 90 s, and 72° C. for 60 s. This profile is termed the “two-phase protocol”. All results shown for cycle number refer to the total number of cycles from the beginning of the PCR (i.e., they include the initial 15 high-temperature cycles). PCRs were performed on a Bio-Rad IQ5 iCycler (software version 2.0.148.60623). DNA was from approximately 50 ml of blood of a healthy volunteer, extracted by the Qiagen QIAamp DNA Blood Maxi Kit according to the manufacturer's protocol (column purification, RNase not used), and quantified by the Invitrogen Qubit fluorometer with the Quant-iT dsDNA BR Assay Kit.

Outer and Inner Primers

The Tₘ values were calculated using a base stacking model (based on Borer et al. (J. Mol. Biol. 86 (1974) 843-853) and SantaLucia (J. SantaLucia Jr., Proc. Natl. Acad. Sci. USA 95 (1998) 1460-1465) and available at http://www.promega.com/biomath/calc11.htm) and assuming 2.5 mM magnesium and 600 nM primer. The range of temperatures over which the primers produced efficient amplification was then determined experimentally using a gradient of annealing temperature. Primers with a variety of predicted Tₘ values were investigated, and ultimately outer primers with Tₘ values of 77-78° C., which were found to produce efficient amplification at least up to 74° C. annealing, and inner primers with Tₘ values of 58-62° C., which were found to produce efficient amplification up to 61-64° C. annealing, were used.

Antisense Oligonucleotides

These molecules were based on the antisense sequence to the 3′ end of the outer primers. They were designed to have a Tₘ that would result in minimal hybridization to the outer primers during the initial high-temperature phase of the PCR but material hybridization during the later low-temperature phase. They carried 5′ and 3′ tags, each consisting of three nucleotides. Because the outer primer would extend along the 5′ tag after hybridization, the 3′-5′ sequence of the tag was the complement of the 3′-5′ sequence of the template strand immediately downstream of the point at which the native primer would hybridize. This design would result in a 3-base mismatch between the 3′ end of the extended primer and its template, with each mismatch being either a:a, g:g, c:c, or t:t. This would block the outer primer from extending further and, thus, prevent amplification of the target. The sequence of the 3 bases at the 3′ end of the oligonucleotide was such that each potentially hybridizing base of the oligonucleotide was the same as the potentially hybridizing base of the outer primer. Again, this design would result in a 3-base mismatch that would block extension of the oligonucleotide along the outer primer. This strategy prevented production, during the initial high-temperature phase of the PCR, of an unduly long antisense oligonucleotide that had a higher Tₘ and that might exert unwanted inhibition.

The Tₘ value of each antisense oligonucleotide was determined for the core sequence, excluding the 5′ and 3′ tags, and the members of each pair had approximately the same Tₘ. The inhibitory activity of each pair was tested by performing a PCR that contained the outer primers and the antisense oligonucleotides but not the inner primers, using as controls both a complete PCR that contained both sets of primers and the antisense oligonucleotides and a PCR that contained only the outer primers. It was found that pairs of oligonucleotides, with each member of the pair having a Tₘ between 48 and 55° C., resulted in satisfactory inhibition of the outer primers during the low-temperature second phase of the PCR without producing inhibition during the high-temperature first phase. The Tₘ values for the antisense oligonucleotides used in the final protocol were 49-54° C.

Data Reporting and Statistical Tests

Data are reported following the MIQE (minimum information for publication of quantitative real-time PCR experiments) guidelines (Bustin et al. Clin. Chem. 55 (2009) 611-622). Differences were tested with the Student's t test.

Results

The antisense qPCR strategy was investigated for four genes: a 475-bp segment of NRAS (neuroblastoma RAS viral oncogene homologue, geneID 4893, MIM 164790), a 287-bp segment of BCR (breakpoint cluster region, geneID 613, MIM 151410), a 474-bp segment of APC (adenomatous polyposis coli, geneID 324, MIM 611731), and a 184-bp segment of a rearranged immunoglobulin heavy chain (IGH) gene (IGH@, geneID 3492). A number of primers and antisense oligonucleotides were investigated during development of each system, and the primers and oligonucleotides finally used are shown in Table 5.

Preliminary investigations, correlating calculated Tₘ values with function at various annealing temperatures, showed that the individual primers amplified efficiently up to annealing temperatures equal to their calculated Tₘ value plus 5-7° C., pairs of primers amplified efficiently up to an annealing temperature equal to their mean Tₘ value plus 2-4° C., and pairs of antisense oligonucleotides produced measurable inhibition of a pair of outer primers up to an annealing temperature equal to the mean oligonucleotide Tₘ value plus approximately 10° C. A useful criterion for satisfactory inhibition of outer primers by a pair of antisense oligonucleotides was that when the only primers in the PCR were the outer primers, a two-phase PCR protocol was used and the mass of DNA added was such that the cycle threshold (Cₜ) was reached after 6 or 7 low-temperature cycles, and then adding the antisense oligonucleotides increased the Cₜ by 5-10 cycles. In addition to their Tₘ, the magnitude of the inhibitory effect of the antisense oligonucleotides was also found to be influenced by their molar concentration, the duration of annealing, and the annealing temperature. The inhibitory effect of the antisense oligonucleotides was decreased if only one oligonucleotide was used in the reaction, but the inhibitory effect could be satisfactorily increased by using an oligonucleotide with a Tₘ increased by 2-3° C. Omitting the 3′ tag of the antisense oligonucleotide resulted in the variable inhibition of amplification during the initial high-temperature phase of the PCR (data not shown).

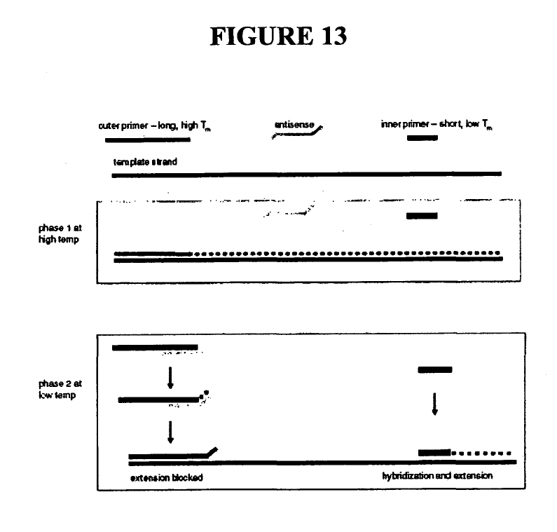

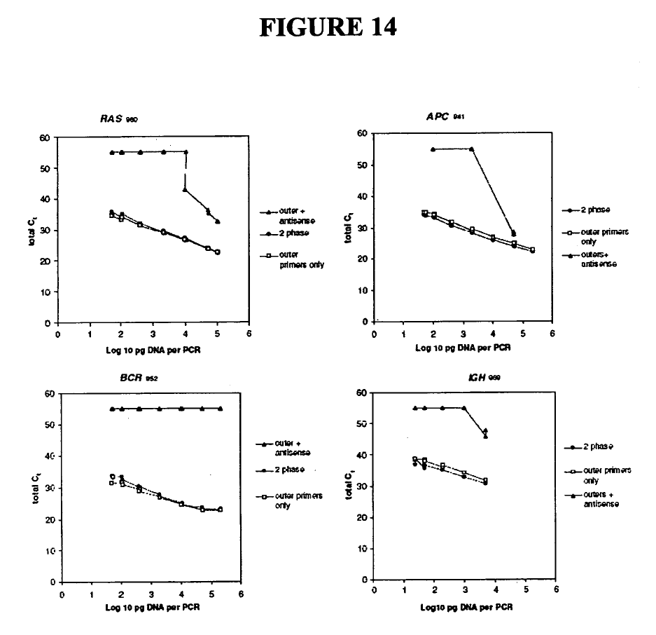

Results using the final antisense qPCR protocol, as described in Materials and methods, are shown in FIG. 14. Amplification of a range of DNA masses was carried out to study performance of the system over a wide range of cycles. It can be seen that the complete system, incorporating outer primers, inner primers, and a pair of antisense oligonucleotides, gave the same Cₜ results as did amplification with the outer primers alone. When the results of amplification in the presence of the outer primers and antisense oligonucleotides are compared with those of amplification by the outer primers only, it is evident that significant inhibition of the outer primers had already occurred after 7 or 8 low-temperature cycles. On the other hand, when the results of amplification in the presence of only the outer primers and antisense oligonucleotides are compared with those of amplification by the complete system, which also contained the inner primers, it is evident that the predominant PCR product after 7 or 8 low-temperature cycles was that which resulted from amplification by the inner primers. Electrophoresis confirmed that the final amplification product was the result of amplification directed by the inner primers, and results from one experiment are shown in FIG. 15.

A direct comparison between amplification efficiency of antisense qPCR and that of standard qPCR was performed in 21 experiments—8 with N-RAS, 5 with BCR, 6 with APC, and 2 with IGH—with each experiment amplifying 0.1-100 ng of DNA. No statistically significant differences were detected. The mean slopes of the relationship between Ct and logarithm of the mass of DNA were −3.62 for antisense qPCR and −3.58 for standard PCR, with the standard error for the paired differences being 0.065. The mean difference between the Ct value for antisense qPCR and that for standard qPCR for any given mass of DNA was −0.467, with the standard error being 0.252 (t=1.85, ^{P<}0.1, two-tailed). The most obvious interpretation of the results is that there is no real difference in amplification efficiency between the two techniques. If there was in fact a small real difference, then the results would actually suggest that amplification by antisense qPCR is more efficient.

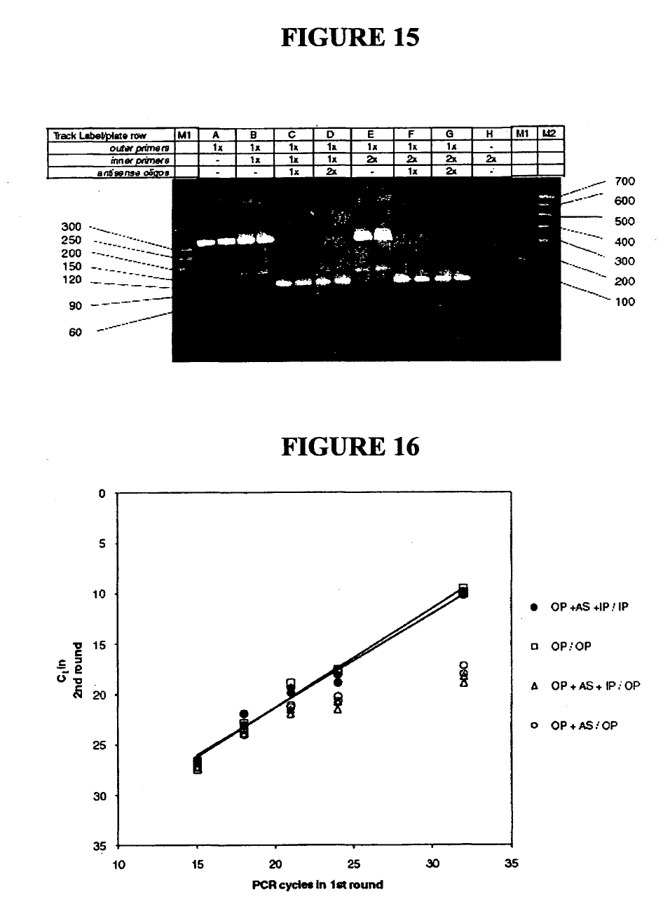

In one experiment studying the APC gene to analyze the results of amplification by the outer and inner primers during antisense PCR, the PCR was stopped after a variable number of cycles and an aliquot was assayed by a second qPCR to quantify the various amplicons produced. In this secondary assay, the outer primers quantified only the long amplicons produced by the outer primers in the primary PCR, whereas the inner primers quantified the total amplicons (i.e., both the short amplicons produced by the inner primers and the long amplicons produced by the outer primers). The results are shown in FIG. 16. Note that the Cₜ scale is shown inversely because the Cₜ value is linearly related to the logarithm of amplicon number. There was an equal and exponential increase in the total number of amplicons produced both by antisense qPCR and by standard qPCR that used only the outer primers. However, when the antisense oligonucleotide was present, either in the antisense PCR or in conjunction with the outer primers only, amplification of long product by the outer primers showed progressive inhibition. This was evident by cycle 21 (i.e., after 6 low-temperature cycles), at which point in the nested PCR only approximately 10% of total amplicons were long amplicons.

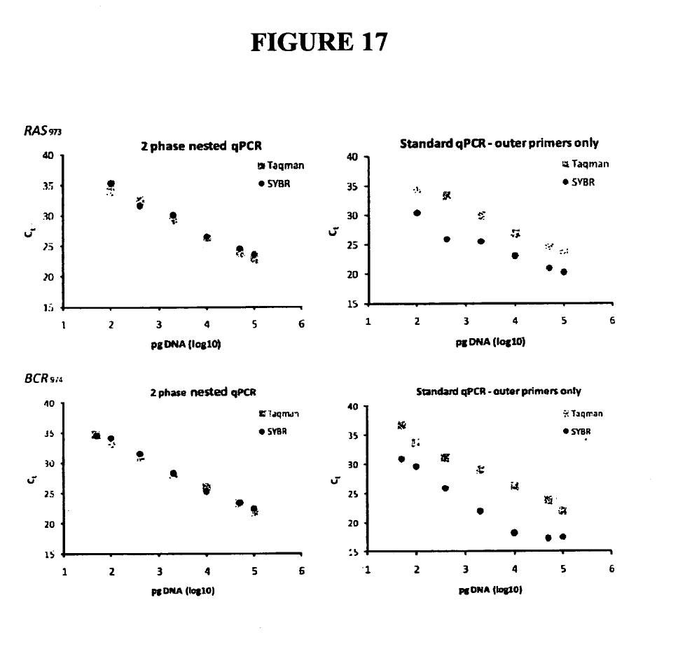

The effect of antisense qPCR on nonspecific amplification was studied for each of the four gene targets. Masses of DNA ranging from 30 pg to 100 ng were amplified either by standard PCR, using the outer primers only, or by antisense qPCR and Cₜ values that were determined either by a Taqman probe, which is specific for the intended product, or by SYBR Green, which measures all PCR products—both the intended product and nonspecific products. In a first experiment with each gene, the two-phase temperature profile was used for both antisense qPCR and standard PCR (using outer primers only). Because the lower annealing temperature after cycle 15 might have been slightly suboptimal for the outer primers alone, a second comparison was performed with N-RAS, APC, and BCR in which antisense qPCR was performed with the usual temperature profile, whereas standard PCR was performed with an annealing temperature of 72° C. for all cycles. Study of amplification of the IGH gene using an annealing temperature of 72° C. for all cycles was not possible because annealing of the Taqman probe decreased above a temperature of 61° C. The results were essentially the same for all seven experiments, two of which are shown in FIG. 17. Antisense PCR resulted in a mean Ct increase of 2.98 (standard error [SE]=0.66, ^{P<}0.01) when compared with PCR with the outer primers only. This increase in the number of cycles taken to reach threshold when antisense PCR was used could have been due to a decrease in specific amplification or to a decrease in nonspecific amplification. However, as shown by the results with Taqman, antisense PCR did not produce a decrease in specific amplification. It actually produced a mean decrease of 0.82 Cₜ units (SE=0.36); that is, threshold was reached slightly earlier with antisense PCR than with classical PCR. Thus, by exclusion, the effect of antisense PCR on the SYBR Green endpoint must have been to decrease non-specific amplification.

Quantification of rare targets requires a large number of PCR cycles, and in this situation it might be desired to perform more than 15 high-temperature cycles so as to decrease the number of low temperature cycles and, hence, minimize the possibility of nonspecific amplification during this phase. Therefore, we investigated whether progressive inactivation of the outer primers by the antisense oligonucleotides during the high-temperature phase might eventually reduce amplification efficiency. Three experiments were performed: one each studying N-RAS, BCR, and APC. Using an annealing temperature of 72° C., various masses of DNA were amplified by the outer primers so that Ct values of up to 35 cycles were observed. When antisense oligonucleotides were also present, there was no inhibition except in the experiment studying N-RAS, where two pairs of antisense oligonucleotides, one with a Tₘ value of 50° C. and one with Tₘ values of 55° C., were tested. Slight inhibition was observed after 32 cycles when the latter pair of primers was used. The data, therefore, suggested that up to 30 cycles could safely be used for the high-temperature first phase of the PCR without measurable inhibition by the antisense oligonucleotides.

TABLE 1
Results of experiment 1024 and 1025, showing inactivation of
active primers and activation of inactive primers during the
course of a PCR. Row A is the control using the active outside
primers only, Row B shows the prolongation achieved by adding
the antisense oligonucleotides to the reaction. Row C shows
that, despite the outer primers being inactive, the addition
of active inner primers can maintain amplification. However
row D, in which inactive rather than active inner primers were
added, once again shows impairment of the reaction. Rows E and
F show that the inactive inner primers can be rendered active
by addition of antisense oligonucleotides directed to them.
Rows E and F differ only in that the antisense oligonucleotide
used in row F had a slightly longer tag.

| | | Ct values | Ct values |
|---|---|---|---|
| | Components | Expt 1024 | Expt 1025 |
| A | outside primers (OP), 100 ng each | 26.7, 26.7 | 26.9, 26.8 |
| B | OP | >45, >45 | >45, >45 |
| | + antisense oligonucleotides to OP, | | |
| | equimolar to OP | | |
| C | OP | 26.1, 28.4 | 28.8, 28.2 |
| | + antisense oligonucleotides to OP, | | |
| | equimolar to OP | | |
| | + inner primers (IP), 200 ng each | | |
| D | OP | >45, >45 | >45, >45 |
| | + antisense oligonucleotides to OP, | | |
| | equimolar to OP | | |
| | + blocked IP, 200 ng each | | |
| E | OP | 27.6, 27.1 | 29.0, 28.1 |
| | + antisense oligonucleotides to OP, | | |
| | equimolar to OP | | |
| | + blocked IP, 200 ng each | | |
| | + antisense oligonucleotides to IP | | |
| | with 4 bp tag, equimolar to IP | | |
| F | OP | 27.8, 27.1 | 26.6, 25.0 |
| | + antisense oligonucleotides to OP, | | |
| | equimolar to OP | | |
| | + blocked IP, 200 ng each | | |
| | + antisense oligonucleotides to IP | | |
| | with 6 bp tag, equimolar to IP | | |

TABLE 2
Primers and antisense oligonucleotides used in experiments 1024 and 1025. See
Table 1 for interpretation of A-F. The pair of sequences shown in each row
are the important variables for that reaction.

| | forward primers and antisense | | reverse primers and antisense | | master |
|---|---|---|---|---|---|
| | oligonucleotides | | oligonucleotides | | mix |
| | identifier | sequence | identifier | sequence | ng/ul |
| A | APC1, 38 | cctgcgaagtacaaggatgccaatat | APC4, 39 | agaactatctaagcttcctctt | 33 |
| | mer | tatgtctcctgg | mer | gatgaagaggagctggg | |
| B | 1CPA | ctcccaggagacataataaac | 4CPA | aatcccagctcctcaag.. | 16.5 |
| | 3-15-3 | | 3-11-3 | | |
| C | APC2-14 | atgagatgccttgg | apc3-17 | catctcttcatgtta | 66 |
| D | APC2-15 | gatgagatgccttggct | apc3-18 | ccatctcttcatgttagggg | 66 |
| | blocked | | blocked | | |
| E | 2CPA15bl | taagagccaaggcatgag | 3CPA18bl | ttgtcccctaacatgaagtct | 101 |
| | 3-15-2-4 | | 3-18-2-4 | | |
| F | 2CPA15bl | tttaagagccaaggcatgag | 3CPA18bl | ttttgtcccctaacatgaagtct | 109 |
| | 3-15-2-6 | | 3-18-2-6 | | |

TABLE 3
Working sheet for reagents for experiments 1024
and 1025. The same protocol was used for both

| | ul | | column master |
|---|---|---|---|
| | each | bulk | mix |
| Platinum taq 1x | 0.2 | 13.2 | 5 wells/col |
| 10 x buffer | 2.5 | 165 | |
| MgCl2, 2.5 mM | 1.25 | 82.5 | |
| dNTP, 2x | 0.5 | 33 | |
| Taqman 1/10 | 0.2 | 13.2 | |
| DNA, 20 ng/ul | 1 | 66 | |
| water | 3.35 | 221.1 | |
| | 9 | 594 | 45 |
| primer mix 1 | 4 | | 20 |
| primer mix 2 | 4 | | 20 |
| primer mix 3 | 4 | | 20 |
| primer mix 4 | 4 | | 20 |
| | 25 | | 125 |
| | 66 | | |

TABLE 4
PCR cycling conditions for experiments 1024 and 1025
PCR conditions

| degrees C. | secs | rpts |
|---|---|---|
| 96 | 120 | 1x |
| 94 | 15 | 15x |
| 58 | 30 | |
| 72 | 30 | |
| 58 | 300 | 1x |
| 94 | 15 | 40x |
| 58 | 90 | |
| 72 | 60 | |

TABLE 5
Primers and their Tm values for the four genes studied.

| | | | Tm |
|---|---|---|---|
| Gene | Primer | Sequence | (° C.) |
| N-RAS | N-RAS out 1 | ctgattactggtttccaacaggttcttgctggtgt | 77 |
| | N-RAS out 2 | cccatacaatcagacagtctcgctactatggcctg | 77 |
| | N-RAS anti 1 | AAGacaccagcaagTTG | 49 |
| | N-RAS anti 2 | TGTcaggccatagtagGCT | 51 |
| | N-RAS in 1 | Tgagtacaaactggtgg | 61 |
| | N-RAS in 2 | Tcacctctatggtggg | 60 |
| | N-RAS probe | vic-attgtcagtgcgcttttcccaacaccacctg- | |
| | | mgb-nfq | |
| APC | APC out 1 | cctgcgaagtacaaggatgccaatattatgtctcctgg | 77 |
| | APC out 2 | agaactatctaagcttcctcttgatgaagaggagctg | 77 |
| | | gg | |
| | APC anti 1 | CTCccaggagacataataAAC | 50 |
| | APC anti 2 | AATcccagctcctcAAG | 51 |
| | APC in 1 | Gatgagatgccttgg | 58 |
| | APC in 2 | Ccatctcttcatgttagg | 59 |
| | APC probe | vic-aagcagaattagatgctcagcactt-mgb-nfq | |
| BCR | BCR out 1 | gttggagaccagcctgaccaacatggtg | 77 |
| | BCR out 2 | caaccccattccccaacaatccctggtaag | 77 |
| | BCR anti 1 | AAAcaccatgttggtcaCCG | 54 |
| | BCR anti 2 | TTCcttaccagggattgtACC | 54 |
| | BCR in 1 | Aatcacaactgcttgg | 59 |
| | BCR in 2 | Cattttgctgtttctagg | 58 |
| | BCR probe | vic-ggttgcagtgagccgagcttgtg-mgb-nfq | |
| D2.02-J6 IGH | IGH out 1 | gcaggagaactagccagagacagcaagagg | 78 |
| rearrangement | IGH out 2 | gcaggaaaccccacaggcagtagcag | 77 |
| | IGH anti 1 | AGGcctcttgctgtcAGA | 51 |
| | IGH anti 2 | AAActgctactgccACA | 50 |
| | IGH in 1 | Attaggggagtggct | 60 |
| | IGH in 2 | Aggccctagagtgg | 60 |

Note.
The 5′ and 3′ tags of the antisense primers are shown in uppercase. The genes are NRAS (neuroblastoma RAS viral oncogene homologue, geneID 4893, MIM 164790), BCR (breakpoint cluster region, geneID 613, MIM 151410), APC (adenomatous polyposis coli, geneID 324, MIM 611731), and a rearranged immunoglobulin heavy chain (IGH) gene (IGH@, geneID 3492).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Borer P. N. et al. (1974) J. Mol. Biol. 86:843

Brisco, M. J., Bartley, P. A., and Morley, A. A. 20 11 Antisense PCR: A simple and robust method for performing nested single-tube PCR. Analytical Biochemistry 409:176-182

Bustin et al. Clin. Chem. 55 (2009) 611-622

Cheng et al., 1994, Effective amplification of long targets from cloned inserts and human genomic DNA. Proc Natl Acad Sci. 91:5695-5699

Ching J et al, 2006, Closed-system multi-stage nucleic acid amplification reactions United States Patent Application 20060177844

Du Breuil Lastrucci, et at 2007 Nested PCR employing degradable primers U.S. Pat. No. 7,273,730

Erlich H A, et al 1994 Methods for nucleic acid amplification U.S. Pat. No. 5,314,809

Grosz R et al 1994 Method for amplification of targeted segments of nucleic acid using nested polymerase chain reaction U.S. Pat. No. 5,340,728

Kemp D J et al, 1990, Simplified colorimetric analysis of polymerase chain reactions: detection of HIV sequences in AIDS Gene, 94: 223-228

Sambrook and Russel, 2001, Molecular Cloning: A Laboratory Manual, 3^{rd} Ed. Cold Spring Harbor, N.W.: Cold Spring Harbor Laboratory Press. Chapter 8: In vitro Amplification of DNA by the Polymerase Chain Reaction.

SantaLucia, J., 1998 Proc. Nat. Acad. Sci. USA 95:1460

Xu Dingbang Xie 2008 Single tube in situ nested polymerase chain reaction method and its use Publication CN1858219

Yourno J, 1996 Method and apparatus for nested polymerase chain reaction (PCR) with single closed reaction tubes U.S. Pat. No. 5,556,773

1. A method of modulating the functionality of an oligonucleotide of interest, said method comprising:
(i) contacting said oligonucleotide of interest with an antisense oligonucleotide directed to said oligonucleotide of interest and effecting a nucleotide sequence change in either or both of the oligonucleotide of interest or the antisense oligonucleotide wherein said sequence change modulates the functionality of the oligonucleotide of interest; or
(ii) effecting a sequence change in an oligonucleotide complex, which complex comprises an oligonucleotide of interest hybridised to an antisense oligonucleotide and wherein said sequence change is effected in an oligonucleotide which forms part of said complex and modulates the functionality of the oligonucleotide of interest.

2-17. (canceled)

18. The method according to claim 1, wherein said antisense oligonucleotide is DNA.

19. The method according to claim 1, wherein said functionality is the capacity of said oligonucleotide of interest to undergo extension along a target nucleic acid.

20. The method according to claim 19, wherein said antisense oligonucleotide is directed to either a protein or a nucleic acid molecule.

21. The method according to claim 20, wherein said antisense oligonucleotide is directed to a DNA molecule.

22. The method according to claim 20, wherein said oligonucleotide of interest is a primer, aptamer, DNAzyme, ribozyme or RNase.

23. The method according to claim 20, wherein said oligonucleotide of interest is a gene, gene fragment, chromosomal gene translocation breakpoint or DNA produced by nucleic acid amplification.

24. The method according to claim 1, wherein said antisense oligonucleotide is a primer.

25. The method according to claim 1, wherein said method comprises hybridising said oligonucleotide of interest to the 3′ end of said antisense oligonucleotide and facilitating the 3′ extension of said oligonucleotide of interest along said antisense oligonucleotide, wherein the antisense nucleotide sequence along which said oligonucleotide of interest extends generates an extension of said oligonucleotide of interest which is either:
(i) complementary to the nucleotide sequence of the target nucleic acid and thereby renders said oligonucleotide of interest functional; or
(ii) mismatched relative to the nucleotide sequence of the target nucleic acid and thereby renders said oligonucleotide of interest non-functional; or
(iii) complementary to a nucleotide sequence of another region of said oligonucleotide of interest and wherein said extension hybridises to said region of said oligonucleotide of interest thereby forming a stem-loop configuration and rendering said oligonucleotide of interest non-functional.

26. The method according to claim 1, wherein said method comprises:
(i) hybridising an antisense oligonucleotide to the 3′ end of said oligonucleotide of interest and facilitating the 3′ extension of said antisense oligonucleotide along said oligonucleotide of interest thereby rendering said oligonucleotide of interest non-functional; or
(ii) hybridising the 3′ end of an antisense oligonucleotide to the 3′ end of said oligonucleotide of interest and facilitating the 3′ extension of said antisense oligonucleotide along said oligonucleotide of interest and the 3′ extension of said oligonucleotide of interest along said antisense oligonucleotide thereby rendering said oligonucleotide of interest non-functional; or
(iii) contacting an oligonucleotide complex, said oligonucleotide complex comprises an oligonucleotide of interest hybridised to an antisense oligonucleotide, with a primer which hybridises to said antisense oligonucleotide at a region which is 3′ to the region to which the oligonucleotide of interest is hybridised and effecting 3′ extension of said primer wherein extending said primer displaces said oligonucleotide of interest and renders said oligonucleotide of interest functional; or
(iv) extending the linear 3′ strand of a looped antisense oligonucleotide, which 3′ linear strand is hybridised to a 5′ linear strand but which 5′ linear strand is longer than the 3′ linear strand and comprises a single stranded region which is complementary to said oligonucleotide of interest and which is hybridised to said oligonucleotide of interest, wherein extending said 3′ linear strand along said single stranded region displaces said oligonucleotide of interest and renders said oligonucleotide of interest functional; or
(v) selectively degrading the linear antisense strand either of a stem-loop nucleic acid molecule or of a duplex between said linear antisense strand and an oligonucleotide of interest, which degradation renders the complementary linear oligonucleotide of interest strand single-stranded and thereby functional; or
(vi) hybridising oligonucleotide of interest with a low Tm to an antisense oligonucleotide and effecting extension of said oligonucleotide of interest wherein said extended oligonucleotide of interest develops an increased Tm and is thereby rendered functional; or
(vii) contacting an oligonucleotide complex, which oligonucleotide complex comprises an oligonucleotide of interest hybridised to the 3′ end of an antisense oligonucleotide and a second oligonucleotide hybridised to said antisense oligonucleotide 3′ to the region to which the oligonucleotide of interest is hybridised and effecting ligation between said oligonucleotide of interest and said second oligonucleotide wherein the functionality of said oligonucleotide of interest is modulated.

27. The method according to claim 1, wherein said method is used during nucleic acid amplification, single tube nested amplification, PCR, isothermal amplification, the production of single stranded DNA.

28. The method according to claim 1, wherein said method is used to regulate the functionality of an aptamer, a DNAzyme, or a ribozyme.

29. The method according to claim 27, wherein said method for nucleic acid amplification is a method of amplifying a target DNA, said method comprising:
(i) contacting a DNA sample with:
(a) a first forward primer directed to said target DNA and a first reverse primer directed to said target DNA; and
(b) a second forward primer directed to said target DNA wherein said second forward primer is directed to a nucleic acid sequence located downstream to the sequence to which said first forward primer is directed; and
a second reverse primer directed to said target DNA wherein said second reverse primer is directed to a nucleic acid sequence located upstream to the sequence to which said first reverse primer is directed; and
(c) one or more antisense oligonucleotides as defined hereinbefore directed to one or more of said primers wherein the functionality of said primers is thereby modulatable; and
wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable extension of said first primers due to antisense oligonucleotide hybridisation to said first primers.

30. The method according to claim 27, wherein said method for nucleic acid amplification is a method of amplifying a target DNA, said method comprising:
(i) contacting a DNA sample with:
(a) a first forward primer directed to said target DNA and a first reverse primer directed to said target DNA; and
(b) a second forward primer directed to said target DNA wherein said second forward primer is directed to a nucleic acid sequence located downstream to the sequence to which said first forward primer is directed
a second reverse primer directed to said target DNA wherein said second reverse primer is directed to a nucleic acid sequence located upstream to the sequence to which said first reverse primer is directed; and
(c) one or more antisense oligonucleotides as defined hereinbefore directed to one or more of said primers wherein the functionality of said primers is thereby modulatable and which antisense oligonucleotide directed to said first primer comprises a 5′ nucleic acid tag sequence, the complementary nucleotide sequence of which tag is mismatched relative to the nucleotide sequence of the DNA region adjacent to the 5′ end of the hybridisation site for said primer; and
wherein the molecules of parts (b) and (c) can be added to the reaction before, during or after the amplification of step (ii) but before the amplification of step (iii).
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said first primer along said tagged antisense oligonucleotide.

31. The method according to claim 27, wherein said method of nucleic acid amplification is a method of amplifying a target DNA, said method comprising:
(i) contacting a DNA sample with:
(a) a first forward primer directed to said target DNA and a first reverse primer directed to said target DNA; and
(b) a second forward primer directed to said target DNA wherein said second forward primer is directed to a nucleic acid sequence located downstream to the sequence to which said first forward primer is directed
a second reverse primer directed to said target DNA wherein said second reverse primer is directed to a nucleic acid sequence located upstream to the sequence to which said first reverse primer is directed; and
(c) one or more antisense oligonucleotides as defined hereinbefore directed to one or more of said primers wherein the functionality of said primers is thereby modulatable and which antisense oligonucleotide directed to said first primer comprises a 5′ nucleic acid tag sequence, the complementary nucleotide sequence of which tag is complementary relative to the nucleotide sequence of another region of the same primer,
wherein the molecules of parts (b) and (c) can be added to the reaction before, during or after the amplification of step (ii) but before the amplification of step (iii); and
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said first primer along said tagged antisense oligonucleotide.

32. The method according to claim 27, wherein said method for nucleic acid amplification is a method of amplifying a target DNA, said method comprising:
(i) contacting a DNA sample with:
(a) a first forward primer directed to said target DNA and a first reverse primer directed to said target DNA; and
(b) a second forward primer directed to said target DNA wherein said second forward primer is directed to a nucleic acid sequence located downstream to the sequence to which said first forward primer is directed
a second reverse primer directed to said target DNA wherein said second reverse primer is directed to a nucleic acid sequence located upstream to the sequence to which said first reverse primer is directed; and
(c) one or more antisense oligonucleotides as defined hereinbefore directed to one or more of said primers wherein the functionality of said primers is thereby modulatable and one or more of which antisense oligonucleotides hybridise to the 3′ end of said first primer and are extendible in the 3′ direction along said primer,
wherein the molecules of parts (b) and (c) can be added to the reaction before, during or after the amplification of step (ii) but before the amplification of step (iii); and
(ii) amplifying the DNA sample of step (i) under conditions which enable hybridisation and extension of said first primers but which do not enable extension of said second primers; and
(iii) subjecting the DNA sample of step (ii) to conditions which render functional said second primers; and
(iv) amplifying the DNA sample of step (iii) under conditions which enable hybridisation and extension of said second primers but which do not enable amplification by said first primers due to extension of said antisense oligonucleotide along said primer.

33. The method according to claim 27, wherein said method for nucleic acid amplification is a method of amplifying a target DNA t, said method comprising:
(i) contacting a DNA sample with:
(a) a first forward primer directed to said target DNA and a first reverse primer directed to said target DNA; and
(b) a second forward primer directed to said target DNA wherein said second forward primer is directed to a nucleic acid sequence located downstream to the sequence to which said first forward primer is directed
a second reverse primer directed to said target DNA wherein said second reverse primer is directed to a nucleic acid sequence located upstream to the sequence to which said first reverse primer is directed
and which second primers comprise a 3′ tag which is mismatched relative to the nucleotide sequence of the target DNA; and
(c) one or more antisense oligonucleotides as defined hereinbefore directed to one or more of said primers wherein the functionality of said primer is thereby modulatable and one of said antisense oligonucleotide sequences comprises both a 5′ region complementary to the 3′ region of said second primer and an even more 5′ region the complementary sequence of which is complementary relative to the sequence of the target DNA adjacent to the primer binding site,
wherein the molecules of parts (b) and (c) can be added to the reaction before, during or after the amplification of step (ii) but before the amplification of step (iii); and
(ii) amplifying the DNA sample of step (i) under conditions which enable hybridisation and extension of said first primers but which do not enable extension of said second primers; and
(iii) subjecting the DNA sample of step (ii) to conditions which enable hybridisation and extension of said second primers along said antisense oligonucleotide; and
(iv) amplifying the DNA sample of step (iii) under conditions which enable hybridisation and extension of said second primers along the DNA region of interest but which do not enable extension of said first primers due to antisense oligonucleotide hybridisation to said first primers.


SEQUENCE LISTING (39 sequences)

SEQ ID NO: 1 - DNA, 38 bp, Artificial Sequence
  Primer
cctgcgaagtacaaggatgccaatattatgtctcctgg

SEQ ID NO: 2 - DNA, 21 bp, Artificial Sequence
  Primer
ctcccaggagacataataaac

SEQ ID NO: 3 - DNA, 14 bp, Artificial Sequence
  Primer
atgagatgccttgg

SEQ ID NO: 4 - DNA, 17 bp, Artificial Sequence
  Primer
gatgagatgccttggct

SEQ ID NO: 5 - DNA, 18 bp, Artificial Sequence
  Primer
taagagccaaggcatgag

SEQ ID NO: 6 - DNA, 20 bp, Artificial Sequence
  Primer
tttaagagccaaggcatgag

SEQ ID NO: 7 - DNA, 39 bp, Artificial Sequence
  Primer
agaactatctaagcttcctcttgatgaagaggagctggg

SEQ ID NO: 8 - DNA, 17 bp, Artificial Sequence
  Primer
aatcccagctcctcaag

SEQ ID NO: 9 - DNA, 15 bp, Artificial Sequence
  Primer
catctcttcatgtta

SEQ ID NO: 10 - DNA, 20 bp, Artificial Sequence
  Primer
ccatctcttcatgttagggg

SEQ ID NO: 11 - DNA, 21 bp, Artificial Sequence
  Primer
ttgtcccctaacatgaagtct

SEQ ID NO: 12 - DNA, 23 bp, Artificial Sequence
  Primer
ttttgtcccctaacatgaagtct

SEQ ID NO: 13 - DNA, 35 bp, Artificial Sequence
  Primer
ctgattactggtttccaacaggttcttgctggtgt

SEQ ID NO: 14 - DNA, 35 bp, Artificial Sequence
  Primer
cccatacaatcagacagtctcgctactatggcctg

SEQ ID NO: 15 - DNA, 17 bp, Artificial Sequence
  Primer
aagacaccagcaagttg

SEQ ID NO: 16 - DNA, 19 bp, Artificial Sequence
  Primer
tgtcaggccatagtaggct

SEQ ID NO: 17 - DNA, 17 bp, Artificial Sequence
  Primer
tgagtacaaactggtgg

SEQ ID NO: 18 - DNA, 16 bp, Artificial Sequence
  Primer
tcacctctatggtggg

SEQ ID NO: 19 - DNA, 31 bp, Artificial Sequence
  Primer
attgtcagtgcgcttttcccaacaccacctg

SEQ ID NO: 20 - DNA, 38 bp, Artificial Sequence; identical to SEQ ID NO: 1
  Primer

SEQ ID NO: 21 - DNA, 39 bp, Artificial Sequence; identical to SEQ ID NO: 7
  Primer

SEQ ID NO: 22 - DNA, 21 bp, Artificial Sequence
  Primer
ctcccaggagacataataaac

SEQ ID NO: 23 - DNA, 17 bp, Artificial Sequence
  Primer
aatcccagctcctcaag

SEQ ID NO: 24 - DNA, 15 bp, Artificial Sequence
  Primer
gatgagatgccttgg

SEQ ID NO: 25 - DNA, 18 bp, Artificial Sequence
  Primer
ccatctcttcatgttagg

SEQ ID NO: 26 - DNA, 25 bp, Artificial Sequence
  Primer
aagcagaattagatgctcagcactt

SEQ ID NO: 27 - DNA, 28 bp, Artificial Sequence
  Primer
gttggagaccagcctgaccaacatggtg

SEQ ID NO: 28 - DNA, 30 bp, Artificial Sequence
  Primer
caaccccattccccaacaatccctggtaag

SEQ ID NO: 29 - DNA, 20 bp, Artificial Sequence
  Primer
aaacaccatgttggtcaccg

SEQ ID NO: 30 - DNA, 21 bp, Artificial Sequence
  Primer
ttccttaccagggattgtacc

SEQ ID NO: 31 - DNA, 16 bp, Artificial Sequence
  Primer
aatcacaactgcttgg

SEQ ID NO: 32 - DNA, 18 bp, Artificial Sequence
  Primer
cattttgctgtttctagg

SEQ ID NO: 33 - DNA, 23 bp, Artificial Sequence
  Primer
ggttgcagtgagccgagcttgtg

SEQ ID NO: 34 - DNA, 30 bp, Artificial Sequence
  Primer
gcaggagaactagccagagacagcaagagg

SEQ ID NO: 35 - DNA, 26 bp, Artificial Sequence
  Primer
gcaggaaaccccacaggcagtagcag

SEQ ID NO: 36 - DNA, 18 bp, Artificial Sequence
  Primer
aggcctcttgctgtcaga

SEQ ID NO: 37 - DNA, 17 bp, Artificial Sequence
  Primer
aaactgctactgccaca

SEQ ID NO: 38 - DNA, 15 bp, Artificial Sequence
  Primer
attaggggagtggct

SEQ ID NO: 39 - DNA, 14 bp, Artificial Sequence
  Primer
aggccctagagtgg